(19) United States
(12) Patent Application Publication
(10) Pub. No.: US 2017/0274003 A1
(43) Pub. Date: Sep. 28, 2017

(54) BLOCKING PIRB UPREGULATES SPINES AND FUNCTIONAL SYNAPSES TO UNLOCK VISUAL CORTICAL PLASTICITY AND FACILITATE RECOVERY FROM AMBLYOPIA

(71) Applicant: The Board of Trustees of the Leland Stanford Junior University, Stanford, CA (US)

(72) Inventors: Carla J. Shatz, Palo Alto, CA (US); David Bochner, Washington, DC (US); Richard W. Sapp, Menlo Park, CA (US)

(21) Appl. No.: 15/511,140

(22) PCT Filed: Sep. 9, 2015
(86) PCT No.: PCT/US15/49156

§ 371 Date: Mar. 14, 2017

Related U.S. Application Data
(60) Provisional application No. 62/051,189, filed on Sep. 16, 2014.

(51) Int. Cl. A61K 31/7105; C07K 2/00; C07K 14/705; A61K 39/395; A61K 38/17; G01N 33/566

(52) U.S. Cl. CPC A61K 31/7105; A61K 38/1774; G01N 33/566; C07K 14/70503; A61K 39/395; C07K 2/00; C07K 2319/00; C07K 16/00

(57) ABSTRACT

Targeting and disrupting paired-immunoglobulin-like receptor B (PirB) function increases synaptic connectivity and plasticity even after the critical period, and can enable significant structural and functional recovery from amblyopia. Provided are compositions comprising PirB, or LILRB2 (leukocyte immunoglobulin-like receptor 2) polypeptides for disrupting PirB/LILRB2 signaling, and methods of using the compositions for treating disorders associated with reduced synaptic plasticity including amblyopia.

### CROSS REFERENCE

This application is a 371 application and claims the benefit of PCT Application No. PCT/US2015/049156, filed Sep. 9, 2015, which claims benefit of U.S. Provisional Patent Application No. 62/051,189, filed Sep. 16, 2014, which applications are incorporated herein by reference in their entirety.

### BACKGROUND OF THE INVENTION

During postnatal development, the capacity of the brain to undergo experience-dependent changes in synaptic strength and circuit connectivity is dynamically regulated, with plasticity peaking during developmental critical periods and then decreasing with maturation. Critical periods are key times when sensory experience is necessary for normal circuit development and when abnormal experience can generate enduring anomalies in brain structure and function. Ocular dominance (OD) plasticity is a graphic example of experience-driven synaptic and circuit plasticity. Children born with congenital cataract in one eye will suffer amblyopia—a loss of visual acuity—if not corrected early in life.

Monocular visual deprivation (MD) has been used in animal models of amblyopia to understand underlying mechanisms. After a brief period of MD or enucleation (ME) during juvenile life, visually driven responses of neurons in the binocular zone of mammalian primary visual cortex (V1) shift towards the open eye, and cortical territory containing neurons responding to open eye stimulation expands, while closed eye responses weaken and territory shrinks. These effects are maximal around postnatal day 28 (P28) in mice and decrease thereafter; by adulthood little OD plasticity resulting from eye closure can be detected, particularly with shorter periods of deprivation.

Furthermore, a long term period of MD (LTMD) spanning the entire critical period (e.g. P19-47) generates an enduring loss of acuity and cortical function in the deprived eye—even if binocular vision is restored in adulthood. This normal decrease in plasticity by adulthood, although important for stabilizing neural circuits, acts as a barrier to recovery after injury because it limits cortical reorganization, can lock in effects of dysfunctional development, and even opposes acquisition of new learning. If adult neural circuits could be returned to an immature state, critical periods might be effectively re-opened, facilitating recovery after nervous system damage, leading to new treatments for neurodegenerative or developmental disorders, or even enhancing learning in healthy individuals.

### SUMMARY OF THE INVENTION

During critical periods of development, the brain easily changes in response to environmental stimuli, but this neural plasticity declines by adulthood. It is shown herein that PirB actively represses neural plasticity throughout life. Methods of disrupting PirB function include, without limitation, infusion of a soluble PirB ectodomain (sPirB). Neural plasticity is enhanced by this treatment both during the critical period and when PirB function was disrupted in adulthood. Acute blockade of PirB triggers the formation of new functional synapses. In addition, recovery from amblyopia—the decline in visual acuity and spine density resulting from long term monocular deprivation, can be achieved following an effective dose of PirB disruption. Thus, neural plasticity in adult visual cortex is actively repressed and can be enhanced by blocking PirB function.

In some embodiments, the methods comprise administering to the individual an effective amount of an agent to disrupt PirB function, for a period of time sufficient to provide the desired neuronal plasticity, e.g. for up to about 3 days, up to about 5 days, up to about 7 days, up to about 10 days, up to about 14 days, etc., which may be administered continuously, e.g. by pump, daily, semi-weekly, semi-daily, etc.

In some embodiments, the agent comprises a PirB/LILRB2 polypeptide. In some embodiments the full extracellular pirB ectodomain is used, which may optionally comprise tags for ease of purification and/or detection, e.g. his, myc, etc. as known in the art, which tags may be removed from the finished product. Such extracellular domain polypeptides comprise 6 Ig domains of the PirB/LILRB2 polypeptide. In some embodiments the PirB/LILRB2 polypeptide consists essentially of the six Ig-like domains of PirB or LILRB2. In some embodiments, the PirB/LILRB2 polypeptide comprises the first two Ig-like domains of PirB or LILRB2. In some embodiments, the PirB/LILRB2 polypeptide consists essentially of the first two Ig-like domains of PirB or LILRB2. In some embodiments, the agent comprises a dimer of PirB/LILRB2 polypeptides. In some embodiments, each PirB/LILRB2 polypeptide of the dimer is fused to an Fc domain. In some embodiments, each PirB/LILRB2 polypeptide of the dimer comprises the first two Ig-like domains of PirB or LILRB2. In some embodiments, each PirB/LILRB2 polypeptide of the dimer consists essentially of the first two Ig-like domains of PirB or LILRB2. In some embodiments, the agent is an antibody that binds to amino acid residues within the first or second Ig-like domain of PirB or LILRB2. In other embodiments, the agent inhibits Aβ oligomer activation of PirB/LILRB2 by inhibiting Aβ oligomer-induced PirB/LILRB2 activation of downstream proteins. In some embodiments, the downstream protein is cofilin, PP2A, PP2B or PP2C.

In some aspects of the invention, compositions are provided, the compositions finding use in the methods of the invention. In some embodiments, the composition comprises the full extracellular pirB ectodomain, which may optionally comprise tags for ease of purification. Such extracellular domain comprises 6 Ig domains of the PirB/LILRB2 polypeptide. In some embodiments the PirB/LILRB2 polypeptide consists essentially of the six Ig-like domains of PirB or LILRB2. The compositions may be formulated with a pharmaceutically acceptable excipient.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1E: A Tamoxifen-inducible Cre-dependent strategy for deletion of PirB with temporal control FIG. 1A: Schematic of PirB protein structure (top) and floxed PirB allele (bottom) before and after Cremediated excision. FIG. 1B: Daily tamoxifen given via injection of nursing mother (P3 to P7) induces deletion of the floxed region at P21 as detected by PCR. FIG. 1C: Western Blots for PirB protein in forebrain at ages (left) of tamoxifen administration and western blotting. FIG. 1D: Quantification of PirB protein in forebrain after tamoxifen administration (P3 to P7), normalized to average Cre− levels across all ages assayed: Cre− P21: n=4 mice vs. Cre+ P21: n=5, p=0.02, U-test. Cre− P27: n=5 vs. Cre+ P27: n=4, p=0.02, U-test. FIG. 1E: Quantification of PirB protein in forebrain at P70 (adult) after tamoxifen injection from P45 to P49. Cre− P70: n=4 mice vs. Cre+ P70: n=4. p=0.03, U-test. * p<0.05.

FIG. 2A-2I: Timed genetic deletion of PirB enhances OD plasticity FIG. 2A: Schematic of mouse visual system. Each retina (right: red, left: blue) projects primarily contralaterally to the lateral geniculate nucleus (LGN), which projects to visual cortex (V1). A small binocular zone (BZ, purple) in V1 receives input from both eyes; in response to deprivation of one eye (e.g. left), the representation of the open (right; ipsilateral) eye expands (arrows). FIG. 2B: Timeline of inducible knockout of PirB and assessment of OD plasticity via Arc mRNA induction. FIG. 2C: Example micrographs of in situ hybridizations for Arc mRNA induced in BZ of visual cortex following open eye stimulation. Each black dot is a cell. Monocular enucleation (ME) from P28 to P32 results in expansion of the ipsilateral (open) eye representation (between red asterisks), as compared with normal rearing (NR). Cre−=PirB flox/flox. Cre⁺=UbC-CreERT2; PirB flox/flox. Width of Arc signal in L4 was measured. Cortical layers indicated at left; scale bar=500 μm. FIG. 2D: Cumulative histograms of width of Arc mRNA signal by individual section. NR Cre−: n=41 sections; NR Cre+: n=44; ME Cre−: n=39; ME Cre+: n=52. FIG. 2E: Graph of data in D, with mean and SEM by animal: deletion of PirB during the critical period enhances OD plasticity. NR Cre−: n=7 mice vs. NR Cre+: n=7, p=0.65; ME Cre−: n=7 vs. ME Cre+: n=7, **** indicates p<0.0001, Two-way ANOVA with Tukey posthoc test. FIG. 2F: Timeline of inducible deletion of PirB and assessment of OD plasticity in adults. FIG. 2G: Example Arc mRNA in situ hybridization micrographs at P74, as in C. FIG. 2H: Cumulative histograms of width of Arc mRNA induction from individual sections. NR Cre−: n=23 sections; NR Cre+: n=20; ME Cre−: n=41; ME Cre+: n=51. FIG. 2I: Graph of data shown in G (mean and SEM by animal): deletion of PirB in adulthood enhances OD plasticity. NR Cre−: n=5 mice vs NR Cre⁺: n=5, p=0.99; ME Cre−: 7 vs ME Cre+: n=8, p=0.013; ME Cre− vs NR Cre−: p=0.18; ME Cre+ vs NR Cre+: p=0.0004, by Two-Way ANOVA with Tukey post-test. * p<0.05, *** p<0.001.

FIG. 3A-3C: Cre-mediated deletion of PirB from forebrain excitatory neurons enhances adult OD plasticity. FIG. 3A: Genotyping of samples from ear and cerebral cortex from P100 CamKIIa-Cre; PirB flox/flox (cKO) or CamKIIa-Cre; PirBWT (WT), showing deletion of floxed PirB in cortex but not ear. FIG. 3B: CamKIIa-Cre; PirB flox/+ breeders were crossed with the Ai14 TdTomato reporter line, generating red fluorescence in the presence of Cre. Sagittal section through visual cortex (layers indicated at right) and hippocampus of a P30 mouse shows Cre present in pyramidal neurons. FIG. 3C: Graphs of width of L4 region activated by stimulation of ipsilateral (open) eye in visual cortex, assessed using Arc mRNA induction. Deletion of PirB from forebrain excitatory neurons increases open eye expansion in adult mice after ME from P100 to 110. NR WT: n=5 mice vs. NR cKO: n=4 mice, p=0.91. ME WT: n=8 mice vs. ME cKO: n=5, p=0.006. NR vs. ME WT: p=0.39, NR vs. ME cKO: p=0.0002, by Two-way ANOVA with Tukey post-hoc test. ** p<0.01, *** p<0.001

FIG. 4A-4J: Blockade of PirB binding enhances OD plasticity in WT visual cortex FIG. 4A: Schematic of soluble PirB-Myc-His (sPirB) fusion protein, indicating extracellular Ig-like domains plus Myc and His tags. FIG. 4B: Western blot of culture supernatant from sPirB-transfected HEK293 cells, detecting Myc tag and PirB ectodomain; Myc-His tagged alkaline phosphatase (AP-Myc-His) is a positive control. FIG. 4C: PirB phosphorylation is decreased after 7 days (P21 to P28) sPirB infusion into WT mouse cortex, as shown by phospho-tyrosine IP and PirB western blot of cortical lysates from infused (sPirB Infused), uninfused (sPirB Uninf) hemispheres, or untreated littermate controls. FIG. 4D: Section of visual cortex immunostained with anti-Myc antibody after 11 day (P21 to P32) sPirB or BSA infusion (1 mg/mL). Scale bar=1 mm. FIG. 4E-4F: Minipump infusions of sPirB during Critical Period (CP). Timeline as shown. FIG. 4E: Example Arc mRNA in situ hybridization micrographs of visual cortex after BSA (top), or sPirB (bottom) treatment. Scale bar=500 μm. Red asterisks indicate borders of Arc mRNA signal induced by stimulating the ipsilateral (open) eye in layer 4. FIG. 4F: Graphs comparing width of Arc mRNA signal in L4 following open-eye stimulation. Width of territory activated by open-eye stimulation following ME is greater following sPirB infusion than with BSA. NR BSA: n=4 mice, NR sPirB: n=4, ME BSA: n=5 vs. ME sPirB: n=6, p<0.0001 by Two-way ANOVA and Tukey post-hoc test for all comparisons indicated. G-I: sPirB infusions into adult WT visual cortex; timeline as shown. FIG. 4G: Example of Arc mRNA situ hybridization micrographs at P74. Scale bar=500 μm. FIG. 4H: Graphs comparing width of Arc mRNA signal in L4 after stimulation of the ipsilateral (open) eye. sPirB infusion from P63 to P74 enhances open-eye expansion following ME. NR BSA: n=4 mice vs. NR sPirB: n=4 p=0.99. ME BSA: n=4 vs. ME sPirB: n=5, p=0.0004. NR vs. ME BSA: p=0.88, NR vs. ME sPirB: p<0.0001. FIG. 4I: sPirB infusion coupled with 3 days of monocular deprivation (MD) also enhances OD plasticity. FIG. 4J: sPirB infusion has no effect on OD plasticity when infused into visual cortex of PirB−/− mice. ME PirB−/− BSA: n=5 mice vs. ME PirB^{-/-} sPirB: n=5, p=0.95, ME PirB−/− BSA vs. ME WT BSA: p=0.034. MD BSA vs MD sPirB n=4 mice/group, p=0.036. * p<0.05, *** p<0.001, **** p<0.0001 by Two-Way ANOVA and Tukey post-hoc test.

FIG. 5A-5E: sPirB increases spine density and functional synapses on L5 pyramidal neurons of normally reared mice. FIG. 5A: Timeline of minipump infusions (1 mg/mL BSA or sPirB from P63 to P74) and example dendrites of YFP-labeled L5 pyramidal neurons in binocular zone of visual cortex in WT Thy1-YFP-H animals reared with normal visual experience. Scale bar=10 μm. FIG. 5B: Histograms of spine density on apical tufts of L5 neurons in sPirB infused vs in the uninfused (Unif.) contralateral hemisphere, or in BSA controls: BSA Infused: n=5 mice vs. sPirB Infused: n=5, p=0.01, 1-2 cells/animal. BSA Uninf.: n=5 vs. sPirB Uninf.: n=5, p=0.96, BSA Inf. vs. Uninf.: p=0.99, sPirB Inf. vs. Uninf.: p=0.016 by Two-Way ANOVA and Tukey post-hoc test. FIG. 5AC: Example traces of mEPSC responses recorded from visual cortical slices (P70 to 77) from L5 pyramidal neurons following BSA or sPirB infusion, as in A. FIG. 5D: Increased mEPSC frequency with sPirB infusion: BSA: n=12 neurons vs. sPirB n=13, p=0.046 by Mann-Whitney U Test. FIG. 5E: No change in mEPSC amplitude: BSA: n=12 neurons vs. sPirB n=13, p=0.70 by Mann-Whitney U Test.

FIG. 6A-6E: sPirB allows structural and functional recovery from amblyopia after LTMD FIG. 6A: Experimental timeline: LTMD from P19 to P47, eye reopening at P47, and minipump infusion from P54 to P61. FIG. 6B: Representative YFP-labeled L5 cell soma and basolateral (arrow) dendrites in visual cortex of WT Thy1-YFP-H mice. Scale bar=50 μm. FIG. 6C: Bar graphs showing changes in basolateral dendritic spine density: LTMD causes a significant decline in spine density (BSA LTMD) that can be fully reversed with sPirB infusion (sPirB LTMD). (BSA NR: n=5 mice vs. BSA LTMD: n=4, p=0.02. sPirB LTMD: n=5, sPirB vs. BSA LTMD, p=0.001, sPirB NR: n=5 animals, 1-2 cells per animal, sPirB vs. BSA NR: p=0.003.) * p<0.05, ** p<0.01 by Two-way ANOVA and Tukey post-hoc test. FIG. 6D: Averaged cortical Visual Evoked Potential response amplitudes (microvolts) to stimuli at a range of spatial frequencies (cycles/degree) following LTMD in mice receiving minipump infusion of either sPirB or BSA. Dotted line: semi-logarithmic regression of visual responses. Inset: Population average traces at 0.05 cyc/de. FIG. 6E: Bar graphs showing spatial acuity following LTMD plus infusion of either BSA or sPirB. Gray shaded region indicates mean acuity±SEM of normally reared (NR) controls. Measurements from individual mice are plotted (circles). Loss of acuity with LTMD is reversed following just one week of sPirB infusion. (NR, n=6 mice vs BSA LTMD, n=5 mice, p=0.004. sPirB LTMD, n=4 mice vs BSA LTMD p=0.016)*p<0.05, **p<0.01 by U Test.

FIG. 7A-7D. Example line scan measurements of Arc mRNA in situ hybridization signal in visual cortex induced by stimulation of the ipsilateral eye Screen shots from the NeuroLens image analysis software showing examples of Arc mRNA in situ hybridization analysis of sections from normally-reared (FIG. 7A and FIG. 7B) or ME from P28 to P32 (FIG. 7C and FIG. 7D) Cre− visual cortex at P32 (see FIG. 2C). First, image contrast was reversed to optimize NeuroLens image acquisition. Then following acquisition, dots (FIG. 7A and FIG. 7C: small red dots) were drawn along the L3-4 border, to permit positioning of a line scan (yellow line). Scans were then made along this line and also 30 pixels (105 μm) above and below as indicated by blue and red lines. The full scan extends from the endpoints (orange boxes) of the defined area and measures the width of a continuous patch of hybridization signal in the binocular zone of V1 (red box). FIG. 7B, FIG. 7D are quantification of the line scans shown above, with the spatial average of pixel intensity (yellow) shown over the extent of the scan. Gray line indicates a linear background model, and the red line indicates the width of Arc mRNA above background. This width corresponds to the red box in FIG. 7A, FIG. 7C and is what is measured as the width of Arc mRNA induction.

FIG. 8A-8F. Plasticity indices for genetic or pharmacological disruption of PirB function. Data from all Arc Induction Experiments, presented as a plasticity index, calculated as width of L4 Arc mRNA signal following ME/width of Arc mRNA signal in NR. Index is a measure of the degree to which cortical territory functionally connected to the ipsilateral (open) eye expands following ME over that present in normally reared controls. FIG. 8A: Inducible Cre-mediated PirB deletion during the critical period plus ME from P28 to P32 (p=0.001). FIG. 8B: Inducible Cremediated PirB deletion in adulthood plus ME from P70 to P74 (p=0.001). FIG. 8C: CamKIIa-Cre; PirBflox conditional deletion in excitatory neurons of the forebrain plus ME from P100-110. FIG. 8D: Minipump infusion of sPirB vs BSA in WT mice during the critical period at P21, plus ME from P28 to P32 (p=0.004). FIG. 8E: Minipump infusion of sPirB vs BSA in WT mice from P63 to P74, plus 4 days ME from P70 to P74 (p=0.016). FIG. 8F: Minipump infusion of sPirB vs BSA in WT mice from P63 to P74, plus 3 days MD from P70 to P73 (p=0.028). All comparisons made via Mann-Whitney U Test.

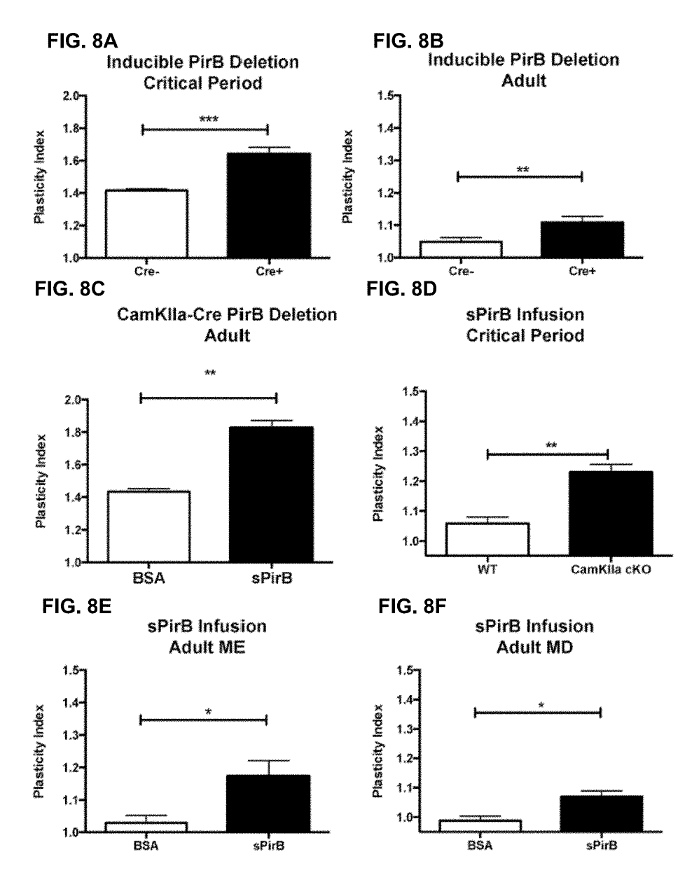

FIG. 9A-9F. Characterization of sPirB minipump infusion area and effect on OD plasticity FIG. 9A: Example minipump implantation site located 2.5 mm lateral and 3 mm posterior to bregma, at anterior tip of V1. FIG. 9B: Anti-Myc immunostain in serial cryosections (16 μm thick) from a P32 mouse after sPirB osmotic minipump infusion, showing diffusion of sPirB caudally into undamaged tissue 2 mm from injection site. FIG. 9C: No Myc immunostaining evident at the injection site or further caudal in BSA-treated controls. Scale bar=1 mm. FIG. 9D: Cumulative histograms of width of Arc mRNA induction in L4 data by individual section from critical period sPirB minipump experiment shown in FIG. 4E, F. sPirB or BSA was infused from P21 to P32 and ME was performed from P28 to P32. NR BSA: 4 animals/23 sections, NR sPirB: 4 animals/32 sections, ME BSA: 5 animals/50 sections, ME sPirB: 6 animals/68 section. FIG. 9E: Cumulative histograms of width of Arc mRNA induction by individual section from adult sPirB experiment shown in FIG. 4F. sPirB or BSA was infused from P63 to P74 and mice were either normally reared (NR) or received ME from P70-74. NR BSA: 4 animals/20 sections, NR sPirB: 4 animals/21 sections, ME BSA: 4 animals/30 sections, ME sPirB: 5 animals/35 sections. FIG. 9F: Cumulative histograms of width of Arc mRNA induction by individual section from experiment shown in FIG. 4G in which sPirB or BSA was infused in visual cortex of PirB−/− (KO) mice from P63-74, coupled with ME from P70-74. ME BSA: n=5 animals/25 sections; ME sPirB: 5 animals/28 sections.

FIG. 10A-10B. Effect of minipump infusions of sPirB or BSA on dendritic spines by cells and by spine type FIG. 10A: Spine density data shown by individual cell from experiment shown in FIG. 5B. Changes in spine density resulting from minipump infusions of sPirB or BSA (1 mg/mL) from P63 to P74 on L5 apical tufts studied in Thy-1 YFP-H WT visual cortex (from FIG. 5B). Data shows spine density on an individual cell basis. See FIG. 5G for analysis per animal. Uninf=uninfused hemisphere contralateral to minipump implantation. ** indicates p<0.01; *** indicates p<0.001 by Two-way ANOVA with Tukey post-test. n=5, 5, 9, 10 YFP-labeled cells. FIG. 10B: Breakdown by spine type: 11 days of sPirB infusion does not alter distribution of spine types. Data is same experiment as in A. FIG. 10C: Spine density changes by cell on L5 basolateral dendrites after normal rearing (NR) or long-term monocular deprivation (LTMD) plus subsequent minipump infusion of sPirB or BSA (from FIG. 5E-G). ** indicates p<0.01, *** indicates P<0.001, and **** indicates P<0.0001 by Two-way ANOVA with Tukey post-test. n=9, 9, 9, 10 cells. FIG. 10D: 7 days of sPirB infusion has no effect on spine morphology on basolateral dendrites of L5 pyramidal neurons after LTMD; same sample as in FIG. 10C.

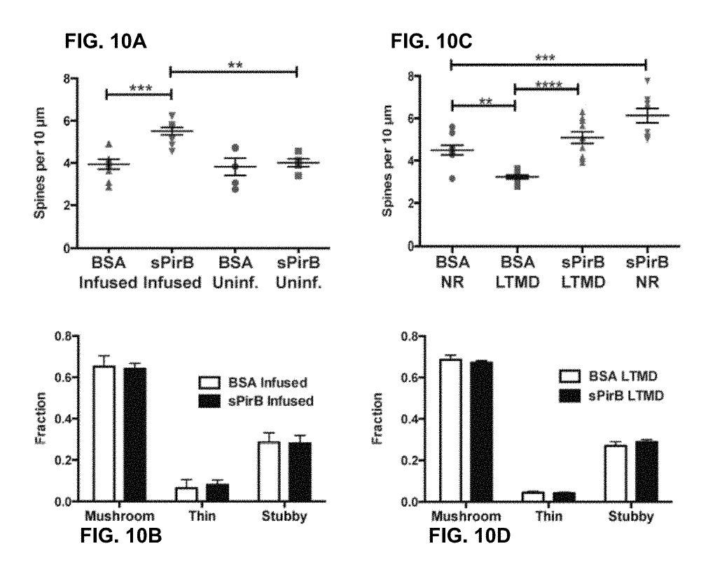

### DETAILED DESCRIPTION OF THE INVENTION

Methods and compositions are provided for disruption of PirB to increase neural plasticity. These methods find many uses, for example, in treating amblyopia, in treating the decline in CNS function in individuals suffering from an Aβ-associated disease or disorder, and for screening candidate agents to identify new therapeutics. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Compositions

Without wishing to be bound by theory, as demonstrated by the working examples below, the present inventors have discovered that by disrupting PirB/LilrB2 signaling, synapse loss may be mitigated and synaptic plasticity restored, thereby permitting and even promoting the formation of new synapses. Accordingly, in some embodiments, the subject compositions inhibit binding to PirB/LILRB2 on the surface of a cell, such that the cell's PirB/LILRB2 receptors do not become activated.

Soluble PirB/LILRB2 polypeptides may be used to disrupt PirB. PirB was first described by Kubagawa et al., Proc. Nat. Acad. Sci. USA 94:5261-6 (1997). Mouse PirB has several human orthologs, which are members of the leukocyte immunoglobulin-like receptor, subfamily B (LILRB), and are also referred to as “immunoglobulin-like transcripts” (ILTs). The human orthologs show significant homology to the murine sequence, from highest to lowest in the following order: LILRB3/ILT5, LILRB1/ILT2, LILRB5/ILT3, LILRB2/ILT4. LILRB3/ILT5 (Genbank Accession No. NP_006855) and LILRB1/ILT2 (Genbank Accession No. NP_006660) were first described by Samaridis and Colonna, Eur. J. Immunol. 27(3):660-665 (1997). LILRB5/ILT3 (Genbank Accession No. NP_006831) was identified by Borges et al., J. Immunol. 159(11):5192-5196 (1997). LILRB2/ILT4/MIR10 (Genbank Accession Nos. NM_005874 (variant 1) and NM_001080978.2 (variant 2)), was identified by Colonna et al., J. Exp. Med. 186:1809-18 (1997). PirB and its human orthologs show a great degree of structural variability.

As known in the art, PirB/LILRB polypeptides are MHC Class I (MHCI) inhibitory receptors, and are known for their role in regulating immune cell activation (Kubagawa et al., supra; Hayami et al., J. Biol. Chem. 272:7320 (1997); Takai et al., Immunology 115:433 (2005); Takai et al., Immunol. Rev. 181:215 (2001); Nakamura et al. Nat. Immunol. 5:623 (2004); Liang et al., Eur. J. Immunol. 32:2418 (2002)). Additionally, PirB is expressed in subsets of neurons throughout the brain. In mutant mice lacking functional PirB, cortical ocular dominance (OD) plasticity is significantly enhanced at all ages, suggesting PirB's play a role in restricting activity-dependent plasticity in visual cortex (Syken et al. (2006) Science 313:1795-800).

The terms “paired-immunoglobulin-like receptor B” and “PirB” are used herein interchangeably, and refer to a native-sequence, 841-amino acid mouse inhibitory protein of GenBank Acccession No. NP_035225, and its native-sequence homologues in rat and other non-human mammals, including all naturally occurring variants, such as alternatively spliced and allelic variants and isoforms, as well as soluble forms thereof. For further details see, Kubagawa et al., Proc Natl Acad Sci USA 94, 5261 (1997). The terms “PirB gene product”, “PirB polypeptide”, “PirB peptide”, and “PirB protein” are used interchangeably herein to refer to native PirB polypeptides, PirB polypeptide variants, PirB polypeptide fragments and chimeric PirB polypeptides.

The terms “LILRB,” “ILT” and “MIR,” are used herein interchangeably, and refer to all members of the human “leukocyte immunoglobulin-like receptor, subfamily B”, including all naturally occurring variants, such as alternatively spliced and allelic variants and isoforms, as well as soluble forms thereof. Individual members within this B-type sub-family of LILR receptors are designated by numbers following the acronym, such as, for example, LILRB3/ILT5, LILRB1/ILT2, LILRB5/ILT3, and LILRB2/ILT4, where a reference to any individual member, unless otherwise noted, also includes reference to all naturally occurring variants, such as alternatively spliced and allelic variants and isoforms, as well as soluble forms thereof. Thus, for example, “LILRB2,” “LIR2,” and “MIR10” are used herein interchangeably and refer to the 598-amino acid polypeptide of Genbank Accession No. NM_005874, and its naturally occurring variants such as alternatively spliced and allelic variants and isoforms, e.g. Genbank Accession No. NM_001080978.2, as well as soluble forms thereof. For further details, see Martin et al., Trends Immunol. 23, 81 (2002). The terms “LILRB gene product”, “LILRB polypeptide”, “LILRB peptide”, and “LILRB protein” are used interchangeably herein to refer to native LILRB polypeptides, LILRB polypeptide variants, LILRB polypeptide fragments and chimeric LILRB polypeptides.

By “native polypeptide” it is meant a polypeptide found in nature. For example, native PirB polypeptides includes mouse PirB, the sequence for which may be found at GenBank Accession No. NP_035225, as well as PirB homologs that naturally occur in other non-human mammals and naturally occurring PirB variants, e.g. isoforms. Likewise, native LILRB polypeptides include LILRB2, the sequence for which may be found at GenBank Accession No. NP_005865, as well as LILRB2 homologs that naturally occur in humans and naturally occurring LILRB2 variants, e.g. isoforms. By “variant” it is meant a mutant of the native polypeptide having less than 100% sequence identity with the native sequence. For example, a variant may be a polypeptide having 60% sequence identity or more with a full length native PirB, e.g. 65%, 70%, 75%, or 80% or more identity, such as 85%, 90%, or 95% or more identity, for example, 98% or 99% identity with the full length native PirB. Variants also include fragments of a native PirB polypeptide that interact with Aβ oligomers, e.g. a fragment comprising residues 24-224 of PirB or the comparable sequence in a PirB homolog or ortholog. Variants also include polypeptides that have Aβ oligomer binding activity and 60% sequence identity or more with a fragment of a native PirB polypeptide, e.g. 65%, 70%, 75%, or 80% or more identity, such as 85%, 90%, or 95% or more sequence identity, for example, 98% or 99% identity with the comparable fragment of the native PirB polypeptide.

The term “PirB/LILRB” is used herein to jointly refer to the corresponding mouse and human proteins and native sequence homologues in other non-human mammals, including all naturally occurring variants, such as alternatively spliced and allelic variants and isoforms, as well as soluble forms thereof. The term “PirB/LILRB2” is used herein to jointly refer to the mouse PirB protein (GenBank Accession Nos. NM_011095.2 and NP_035225, the human LILRB2 protein (GenBank Accession Nos. NM_005874 and NP_005865), and the corresponding PirB protein in other non-human mammals, including all naturally occurring variants, such as alternatively spliced and allelic variants and isoforms, as well as soluble forms thereof.

As demonstrated in the working examples below, Aβ oligomers, e.g. aggregates of Aβ₁₋₄₀ (Aβ40), Aβ₁₋₄₂ (Aβ42), etc. bind to PirB/LILRB2 in vitro and in vivo. More particular, Aβ oligomers bind to PirB polypeptide comprising the first two Ig-like domains of PirB, i.e. residues 24-224 of SEQ ID NO:1. In humans, Aβ oligomers bind to LILRB2, and more particularly to LILRB2 polypeptides comprising the first two Ig-like domains of LILRB2, i.e. residues 24-223 of SEQ ID NO:3. As such, in some aspects of the invention, the effects of Aβ oligomers on neurons and the impact of Aβ oligomers on CNS function, e.g. cognition or vision, in individuals is inhibited by providing an agent that inhibits PirB/LILRB2 signaling induced by Aβ oligomers, e.g. oligomers of Aβ40 or Aβ42, in PirB/LILRB2-expressing neurons.

By disrupting PirB/LILRB2 it is meant reducing, inhibiting, antagonizing, or blocking, the activity of PirB/LILRB2 by 20% or more, for example by 30% or more, by 40% or more, or by 50% or more, in some instances, by 60% or more, by 70% or more, by 80% or more, e.g. by 90% or more, by 95% or more, or by 100%, i.e. to negligible amounts. Any convenient agent may be used. For example, PirB/LILRB2 activity may be inhibited with PirB/LILRB2-specific antibodies, small molecules that bind to PirB/LILRB2 and inhibit activity, or soluble PirB/LILRB2 polypeptides, e.g. PirB/LILRB2 polypeptide comprising the first two Ig-like domains of PirB (residues 24-224) or LILRB2 (residues 24-223) or variants thereof; PirB/LILRB2 polypeptide comprising the first three Ig-like domains of PirB (residues 24-322) or LILRB2 (residues 24-323) or variants thereof; PirB/LILRB2 polypeptide comprising the first four Ig-like domains of PirB (residues 24-422) or LILRB2 (residues 24-458) or variants thereof; PirB/LILRB2 polypeptide comprising the complete extracellular domain of PirB or LILRB2, for example a PirB/LILRB extracellular domain (ECD) polypeptide or variants thereof, where an ECD polypeptide includes a polypeptide that does not include the PirB/LILRB2 transmembrane region or cytoplasmic domain, or a full-length PirB/LILRB polypeptide or variant thereof.

In some instances, the polypeptide consists essentially of the ECD of PirB or LILRB2. In some embodiments, the composition comprises the full extracellular pirB ectodomain, which may optionally comprise tags for ease of purification. Such extracellular domain comprises 6 Ig domains of the PirB/LILRB2 polypeptide. In some embodiments the PirB/LILRB2 polypeptide consists essentially of the six Ig-like domains of PirB or LILRB2. In some instances, the polypeptide consists essentially of the first four Ig-like domains of PirB or LILRB2 or variants thereof. In some instances, the polypeptides consist essentially of the first three Ig-like domains of PirB or LILRB2 or variants thereof. In some instances, the polypeptides consist essentially of the first two Ig-like domains of PirB or LILRB2 or variants thereof.

In aspects of the invention compositions are provided for inhibiting the effects of Aβ oligomers on cells. By Aβ, or “amyloid beta”, or “amyloid β”, it is meant a peptide of 36-43 amino acids that is derived from the processing of amyloid precursor protein (APP) by β- and γ-secretases. By “Aβ oligomers”, “amyloid β oligomers”, or “amyloid beta oligomers” it is meant aggregates of Aβ peptide. Aβ is the main component of deposits, called amyloid plaques, found in the brains of patients with Alzheimer's disease (AD) and cerebral amyloid angiopathy (CAA); it also associated with retinal ganglion cells in patients having glaucoma. Two major variants, Aβ₁₋₄₀ (“Aβ40”) (DAEFRHDSGYEVHHQKLVFFAEDVGSNKGAIIGLMVGGVV) and Aβ₁₋₄₂ (“Aβ42”) (DAEFRHDSGYEVHHQKLVFAAEDVGSNKGAIIGLMVGGVVIA), are produced by alternative carboxy-terminal truncation of APP (Selkoe et al. (1988) Proc. Natl. Acad. Sci. USA 85:7341-7345; Selkoe, (1993) Trends Neurosci 16:403-409). Aβ₁₋₄₂ is the more fibrillogenic and more abundant of the two peptides in amyloid deposits of both AD and CAA. Other naturally occurring variants include, e.g., Aβ₁₋₂₈ (DAEFRHDSGYEVHHQAAVFAAEDVGSNK), Aβ12-28 (VHHQKLVFFAEDVGSNKC), Aβ₁₋₃₇ (DAEFRHDSGYEVHHQKLVFFAEDVGSNKGAIIGLMVG), Aβ₁₋₃₈ (DAEFRHDSGYEVHHQKLVFFAEDVGSNKGAIIGLMVGG), Aβ₁₋₃₉ (DAEFRHDSGYEVHHQKLVFFAEDVGSNKGAIIGLMVGGV), Aβ₁₋₄₃ (DAEFRHDSGYEVHHQKLVFFAEDVGSNKGAIIGLMVGGVVIAT), Aβ₁₋₄₄ (DAEFRHDSGYEVHHQKLVFFAEDVGSNKGAIIGLMVGGVVIATV), Aβ₁₋₄₅ (DAEFRHDSGYEVHHQKLVFFAEDVGSNKGAIIGLMVGGVVIATVI), Aβ₁₋₄₆ (DAEFRHDSGYEVHHQKLVFFAEDVGSNKGAIIGLMVGGVVIATVIV), Aβ₁₋₄₇ (DAEFRHDSGYEVHHQKLVFFAEDVGSNKGAIIGLMVGGVVIATVIVI), Aβ₁₋₄₈ (DAEFRHDSGYEVHHQKLVFFAEDVGSNKGAIIGLMVGGVVIATVIVIT), Aβ₁₋₄₉ (DAEFRHDSGYEVHHQKLVFFAEDVGSNKGAIIGLMVGGVVIATVIVITL), Aβ₁₋₅₅ (DAEFRHDSGYEVHHQKLVFFAEDVGSNKGAIIGLMVGGVVIATVIVITLVMLKKK), Aβ₂₋₄₀ (AEFRHDSGYEVHHQKLVFFAEDVGSNKGAIIGLMVGGVV), and Aβ₃₋₄₀ (EFRHDSGYEVHHQKLVFFAEDVGSNKGAIIGLMVGGVV), as well as derivatives of the above peptides comprising a naturally occurring substitution, e.g. Aβ₁₋₄₂ H13R, Aβ₁₋₄₂ V18A, Aβ₁₋₄₂ F19P, Aβ₁₋₄₂ E22D, Aβ₁₋₄₂ E22V, Aβ₁₋₄₂ E22A, Aβ₁₋₄₂ D23A, Aβ₁₋₄₂ G25A, Aβ₁₋₄₂ N27A, Aβ₁₋₄₂ K28A, Aβ₁₋₄₂ G29A, Aβ₁₋₄₂ I31A, Aβ₁₋₄₂ G37A, the English Mutation, the Iowa Mutation, the Tottori-Japanese Mutation, the Flemish Mutation, the Arctic Mutation, the Italian Mutation, etc. In addition to the amyloid deposits that may occur in, for example, CNS tissue, amyloid deposition may occur in the vascular walls (Hardy (1997); Haan et al. (1990); Vinters (1987); Itoh et al. (1993); Yamada et al. (1993); Greenberg et al. (1993); Levy et al. (1990)). These vascular lesions are the hallmark of CAA, which can exist in the absence of AD.

Aβ oligomers are known in the art to have a number of effects on cells. These include, for example, reducing cell viability, and reducing synaptic plasticity, promoting synapse loss in neurons. By a “synapse” it is meant the structure on a neuron that permits the neuron to pass an electrical or chemical signal to another cell. By “synaptic plasticity” it is meant the ability of the synapse to change in strength, i.e. to become stronger or weaker, in response to either use or disuse, respectively, of transmission over that synaptic pathway. Such a change in strength is typically evident by one or more of the following structural changes: a change in the number of presynaptic vesicles, a change in the amount of neurotransmitter loaded per vesicle, a change in the number of dendritic spines, and/or a change in the number of neurotransmitter receptors positioned on the postsynaptic neuron. Reductions or enhancements in synaptic plasticity may be observed by assessing the ability of a postsynaptic neuron to evoke a long-term enhancement (“long term potentiation”, LTP) or long-term depression (LTD) in the activity of a presynaptic neuron, and/or by assaying for the subsequent changes in synaptic strength, e.g. by detecting one or more of the above-mentioned structural changes. By “enhanced synaptic plasticity” it is meant greater synaptic strengthening (LTP), more stable synapses and a failure to remove synapses and the spines that carry synapses. By “reduced synaptic plasticity” it is meant enhanced synaptic weakening (LTD), less stable synapses, and fewer spines and synapses. By “synapse loss” it is meant a decrease in the number of synapses, for example, a loss in the connection between two neurons or, in instances in which multiple synapses exist between two neurons, in the loss of one or more of these synapses. As is well known in the art, synaptic activity and the change in the strength and number of synapses is central to almost all neurobiological processes, including learning, memory, and neuronal development. In further describing aspects of the invention, the following description focuses on the effects of Aβ oligomers on neurons. However, the subject methods and compositions also find use in inhibiting the effects of Aβ oligomers on other types of cells as well, for example, microglia.

By “comprising” it is meant that the recited elements are required in the composition, method, or kit, but other elements may be included to form the composition/method/kit etc. within the scope of the claim. For example, a polypeptide that comprises PirB/LILRB2 amino acid sequence corresponding to, e.g. residues 24-223 of SEQ ID NO:3 or, e.g., residues 24-458 of SEQ ID NO:3, may comprise LILRB2 amino acid sequence in addition to that sequence with the exception of any sequence recited by negative provisos. By “consisting essentially of”, it is meant a limitation of the scope of composition or method described to the specified materials or steps that do not materially affect the basic and novel characteristic(s) of the subject invention. For example, a PirB/LILRB2 polypeptide “consisting essentially of” a disclosed sequence has the amino acid sequence of the disclosed sequence plus or minus about 5 amino acid residues at the boundaries of the sequence based upon the full length parent PirB/LILRB2 sequence from which it was derived, e.g. about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue. By “consisting of”, it is meant the exclusion from the composition, method, or kit of any element, step, or ingredient not specified in the claim. For example, a PirB/LILRB2 polypeptide “consisting of” a disclosed sequence consists only of the disclosed amino acid sequence. Compositions comprising a polypeptide “comprising”, “consisting essentially of”, or “consisting of” a disclosed PirB/LILRB2 sequence may comprise other elements in addition to the PirB/LILRB2 polypeptide(s), e.g. functional moieties such as polypeptides, small molecules, or nucleic acids bound, e.g. covalently bound, to the PirB/LILRB2 polypeptide; agents that promote the dimerization of the PirB/LILRB2 polypeptide; agents that promote the stability of the PirB/LILRB2 composition; agents that promote the solubility of the PirB/LILRB2 composition; adjuvants, etc. as will be readily understood in the art, with the exception of elements that are encompassed by any negative provisos.

Additional agents that disrupt PirB/LILRB2 may be readily identified using well-known techniques in the art or as described below for quantitatively or qualitatively detecting changes in activity, for example, surface plasmon resonance (SPR), immunoprecipitation of PirB/LILRB2 and detection by western blotting, etc.

Also included as non-limiting examples of agents that disrupt downstream targets. PirB/LILRB2 is shown herein to interact with cofilin and the Ser/Thr phosphatases PP2A, PP2B, and PP2C. By disrupting PirB/LILRB2 activity it is meant reducing activation by PirB/LILRB2 of downstream proteins by 20% or more, for example by 30% or more, by 40% or more, or by 50% or more, in some instances, by 60% or more, by 70% or more, by 80% or more, e.g. by 90% or more, by 95% or more, or by 100%, i.e. to negligible amounts. Any convenient agent that inhibits PirB/LILRB2 activity in the presence of Aβ oligomers may be used, for example, PirB/LILRB2 intracellular peptides, e.g. residues 664-841 of NP_035225 (PirB) or residues 483-598 of NP_005865 (LILRB2) or fragments thereof; nucleic acids encoding residues 664-841 of NP_035225 (PirB) or residues 483-598 of NP_005865 (LILRB2) or fragments thereof; small molecules that interfere with binding of PirB/LILRB2 to cofilin and/or dephosphorylation of cofilin by PirB/LILRB2, PP2A, PP2B or PP2C, etc. Agents may be readily identified using any convenient technique for quantitatively or qualitatively detecting changes in PirB/LILRB2 signaling, for example, detecting the phosphorylation state of cofilin by Western blotting, flow cytometry or immunostaining; detecting the interaction of PirB/LILRB2 with cofilin, PP2A, PP2B, or PP2C by immunoprecipitation and Western blotting; assaying actin disassembly by immunohistochemistry, etc.

As indicated above, agents also include small molecule compounds. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992). Small molecule compounds can be provided directly to the medium in which the cells are being cultured, for example as a solution in DMSO or other solvent.

Agents also include polypeptides, for example, polypeptides that bind Aβ oligomers, e.g., PirB/LILRB2 polypeptides comprising the first two Ig-like domains of PirB (residues 24-224 of NP_035225) or LILRB2 (residues 24-223 of NP_005865) or variants thereof, polypeptides comprising the first three Ig-like domains of PirB or LILRB2 (residues 24-323 of NP_005865) or variants thereof, polypeptides comprising the first four Ig-like domains of PirB (residues 24-422 of NP_035225) or LILRB2 (residues 24-458 of NP_005865) or variants thereof, or polypeptides comprising the complete extracellular domain of PirB or LILRB2 and variants thereof. By an ECD polypeptide it is meant a polypeptide that does not include the transmembrane region or cytoplasmic domain. In some instances, the polypeptide consists essentially of the ECD of PirB or LILRB2. In some instances, the polypeptide consists essentially of the first four Ig-like domains of PirB or LILRB2 or variants thereof. In some instances, the polypeptides consist essentially of the first three Ig-like domains of PirB or LILRB2 or variants thereof. In some instances, the polypeptides consist essentially of the first two Ig-like domains of PirB or LILRB2 or variants thereof. Another example of polypeptides of interest as subject agents are PirB/LILRB2 polypeptides that bind cofilin or PP2, e.g. residues 664-841 of NP_035225 (PirB) or residues 483-598 of NP_005865 (LILRB2) or fragments thereof.

In some instances, the PirB/LILRB2 polypeptide or fragment thereof, e.g. as described above, is fused to an Fc domain, e.g. IgG (IgG1, IgG2, IgG3, IgG4), IgD, IgE. In other words, the subject agent is a polypeptide comprising, consisting essentially of, or consisting of a PirB/LILRB2 polypeptide or fragment thereof fused to an Fc domain, e.g human IgG1, IgG2, IgG3, IgG4. In some instances, e.g. when the Fc domain comprises a hinge region, the Fc domain promotes dimerization. In other instances, e.g. when the hinge region of the Fc domain is absent, the Fc domain does not promote dimerization.

Polypeptides for use as agents in the subject compositions may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

The polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide, e.g. greater than about 20 hours, when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The polypeptide agent typically is fused to the plasma protein, e.g. IgG at the N-terminus of the plasma protein or fragment thereof which is capable of conferring an extended half-life upon the polypeptide. Increases of greater than about 100% on the plasma half-life of the polypeptide are satisfactory. Ordinarily, the polypeptide is fused C-terminally to the N-terminus of the constant region of immunoglobulins in place of the variable region(s) thereof, however N-terminal fusions may also find use. Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods.

The site at which the fusion is made may be selected in order to optimize the biological activity, secretion or binding characteristics of the polypeptide. The optimal site will be determined by routine experimentation.

In some embodiments the hybrid immunoglobulins are assembled as monomers, or hetero- or homo-multimers, and particularly as dimers or tetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA immunoglobulin, and occasionally IgG immunoglobulin, may also exist in a multimeric form in serum. In the case of multimers, each four chain unit may be the same or different.

The polypeptide agent for use in the subject compositions may be produced from eukaryotic produced by prokaryotic cells, it may be further processed by unfolding, e.g. heat denaturation, DTT reduction, etc. and may be further refolded, using methods known in the art.

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The subject polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Another example of polypeptide agents are antibodies, e.g. PirB-specific antibodies that bind to the extracellular domain of PirB, e.g. the PirB specific antibody disclosed in US Publication No. 2009/0285803, the disclosure of which is incorporated herein by reference. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” The term “antibody” herein is used in the broadest sense and specifically covers intact antibodies, monoclonal antibodies, polyclonal antibodies, multispecific antibodies (e.g. bispecific antibodies) formed from at least two intact antibodies, and antibody fragments, so long as they exhibit the desired biological activity. Antibodies are typically provided in the media in which the cells are cultured.

Agents suitable for the subject methods also include nucleic acids, for example, nucleic acids that encode siRNA, shRNA or antisense molecules of PirB or LILRB2, or nucleic acids that encode polypeptides, e.g. PirB/LILRB2 polypeptides comprising or consisting essentially of PirB/LILRB2 extracellular domain sequences that bind Aβ oligomers, e.g. PirB/LILRB2 cytoplasmic tail sequences that bind cofilin or Ser/Thr phosphatases, PP2A, PP2B or PP2C, e.g. nucleic acids encoding residues 664-841 of NP_035225 (PirB) or residues 483-598 of NP_005865 (LILRB2) or fragments thereof. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc.

Nucleic acid may be provided directly to cells. In other words, the cells, e.g. neurons, are contacted with vectors comprising the nucleic acid of interest such that the vectors are taken up by the cells. Methods for contacting cells with nucleic acid vectors, such as electroporation, calcium chloride transfection, and lipofection, are well known in the art.

Alternatively, the nucleic acid of interest may be provided to the subject cells via a virus. In other words, the cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the subject nucleic acid agent into packaging cell lines and of collecting the viral particles that are generated by the packaging lines are well known in the art.

Vectors used for providing nucleic acid of interest to cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing the subject agent to cells may include nucleic acid sequences that encode for selectable markers in the target cells, so as to identify cells that have taken up the subject agent.

Agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

For inclusion in a medicament, the subject agent may be obtained from a suitable commercial source. As a general proposition, the total pharmaceutically effective amount of the subject agent administered parenterally per dose will be in a range that can be measured by a dose response curve.

Preparations of subject agent to be used for therapeutic administration must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The subject agent-based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The subject agent can be incorporated into a variety of formulations. More particularly, the subject agent of the present invention can be formulated into pharmaceutical compositions by combination with appropriate pharmaceutically acceptable carriers or diluents.

Pharmaceutical preparations are compositions that include one or more targeted subject agents present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. As such, administration of the subject agent can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood-brain barrier (BBB). One strategy for drug delivery through the blood-brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

### Methods

Also provided are methods for reducing the effects of PirB on a cell, which find many uses in medicine and research, for example in increasing neural plasticity, including visual deficits, reducing cognitive decline or visual decline in an individual suffering from Aβ-associated disease or disorder, and for screening candidate agents to identify new therapeutics.

Cells of interest include cells that express PirB/LILRB2, e.g. PirB or LILRB2-positive cells. By a “PirB/LILRB2-expressing cell” it is meant a cell that transcribes and translates the PirB or LILRB2 gene. In other words, the cell is positive for PirB/LILRB2 expression. A cell may be readily identified as PirB/LILRB2 positive by detecting the expression of LilrB2. Should it be useful to detect PirB/LILRB on the surface of a cell, any convenient technique for detecting LilrB2 on the surface of a cell may be employed, for example, immunohistochemistry, flow cytometry, ELISA, Western blotting, in situ hybridization, etc. Examples of PirB/LILRB2 positive cells include neurons, e.g. retinal ganglion cells and projection neurons of the cerebral cortex, and microglia.

Cells of interest also include cells that do not express PirB/LILRB2, i.e. PirB/LILRB2-negative cells. For example, as discussed above, it is envisioned that the subject compositions, e.g. PirB/LILRB2 polypeptide mimetics, e.g. PirB/LILRB2 polypeptides and variants thereof as described elsewhere herein, may be administered to prevent Aβ oligomers from binding to other Aβ receptors, e.g. the NMDA receptor (Snyder et al. (2005) Nat Neurosci. 8(8):1051-8), cellular prion protein (Laurén J, et al. (2009) Nature 457(7233):1128-32), the receptor tyrosine kinase EphB2 (Cissé M, eta l. (2011) Nature 469(7328):47-52), the receptor for advanced glycation end products (“RAGE”, Origlia N, et al. (2008) J Neurosci. 28(13):3521-30), metabotropic glutamate receptor 5 (Um J W, et al. (2013) Neuron 79(5):887-902), and the immune cell receptor FcγRIIb (Kam T, et al. J Clin Invest. 2013 Jun. 10), e.g. by binding to Aβ oligomers and hindering the binding of Aβ to other (i.e. non-PirB/LILRB2) Aβ receptors on the cell. Such cells may be readily identified by one of ordinary skill in the art, for example by using techniques in the art for detecting the expression of one or more of these other receptors, and/or for detecting the binding of Aβ oligomers to a cell.

In practicing the subject methods of the application, cells, i.e. in vivo or in vitro, are contacted with an effective amount of the agent to inhibit the effects PirB on cells and to the individual. Biochemically speaking, an “effective amount” or “effective dose” of an agent is an amount of agent that will inhibit, antagonize, decrease, reduce, or suppress by about 20% or more, e.g. by 30% or more, by 40% or more, or by 50% or more, in some instances by 60% or more, by 70% or more, by 80% or more, or by 90% or more, in some cases by about 100%, i.e. to negligible amounts, and in some instances reverse, the effects of PirB signaling on a cell. Methods for measuring cell viability and cell death are well known in the art, any of which may be used to determine an effective dose.

As another example, and “effective amount” or “effective dose” of an agent is an amount of agent that will inhibit, antagonize, decrease, reduce, or suppress by about 20% or more, e.g. by 30% or more, by 40% or more, or by 50% or more, in some instances by 60% or more, by 70% or more, by 80% or more, or by 90% or more, in some cases by about 100%, i.e. to negligible amounts, the reduction in synaptic plasticity and loss of synapses that occurs in the presence of PirB. In other words, cells contacted with an effective amount of the agent will become more responsive to cues, e.g. activity cues, which promote the formation and maintenance of synapses.

Methods for measuring synaptic plasticity in individuals are well known in the art, any of which may be used to determine an effective dose. These include, for example, observing the induction of LTP in neural circuits in awake individuals, e.g. by performing non-invasive stimulation techniques on awake individuals to induce LTP-like long-lasting changes in localized neural activity (Cooke S F, Bliss T V (2006) Plasticity in the human central nervous system. Brain. 129(Pt 7):1659-73); mapping plasticity and increased neural circuit activity in individuals, e.g. by using positron emission tomography, functional magnetic resonance imaging, and/or transcranial magnetic stimulation (Cramer and Bastings (2000) Mapping clinically relevant plasticity after stroke. Neuropharmacology. 39(5):842-51); and by detecting neural plasticity following learning, i.e. improvements in memory, e.g. by assaying retrieval-related brain activity (Buchmann A, et al. (2008) Prion protein M129V polymorphism affects retrieval-related brain activity. Neuropsychologia. 46(9):2389-402) or, e.g., by imaging brain tissue by functional magnetic resonance imaging (fMRI) following repetition priming with familiar and unfamiliar objects (Soldan A, et al. (2008) Global familiarity of visual stimuli affects repetition-related neural plasticity but not repetition priming. Neuroimage. 39(1):515-26; Soldan A, et al. (2008) Aging does not affect brain patterns of repetition effects associated with perceptual priming of novel objects. J Cogn Neurosci. 20(10):1762-76). In some aspects of the subject methods, the method further comprises the step of measuring one or more of these effects.

In a clinical sense, an effective amount, or dose, of an agent is an amount of agent that, when administered for a suitable period of time, usually at least about one week, and maybe about two weeks, or more, up to a period of about 4 weeks, 8 weeks, or longer will evidence an alteration in the symptoms associated with reduced synaptic plasticity and synapse loss in a disease of interest, e.g. amblyopia, glaucoma, cognitive impairment in an individual with Alzheimer's Disease, CAA, or Down syndrome relative to a healthy individual, visual impairment in an individual with glaucoma relative to a healthy individual, etc. For example, an effective dose is the dose that when administered for a suitable period of time, usually at least about one week, and maybe about two weeks, or more, up to a period of about 4 weeks, 8 weeks, or longer will slow e.g. by about 20% or more, e.g. by 30% or more, by 40% or more, or by 50% or more, in some instances by 60% or more, by 70% or more, by 80% or more, or by 90% or more, or halt cognitive decline, i.e. stabilize the cognitive abilities, in a patient suffering from Alzheimer's disease, CAA, or Down syndrome or halt visual decline, i.e. stabilize the visual abilities, in a patient suffering from amblyopia or glaucoma. In some embodiments, an effective amount or dose may not only slow or halt the progression of the disease condition but may also induce the reversal of the condition. For example, an effective dose is the dose that when administered for a suitable period of time, usually at least about one week, and maybe about two weeks, or more, up to a period of about 4 weeks, 8 weeks, or longer will improve the cognition in an individual with Alzheimer's Disease, CAA, or Down syndrome, or vision in an individual with amblyopia or glaucoma, by, for example 1.5-fold, 2-fold, 3-fold, 4-fold, 5-fold, in some instances 6-fold, 7-fold, 8-fold, 9-fold, or 10-fold or more. It will be understood by those of skill in the art that an initial dose may be administered for such periods of time, followed by maintenance doses, which, in some cases, will be at a reduced dosage.

Methods for measuring cognition or vision are also well known in the art, any of which may be used to determine an effective dose Examples include tests such as cognition tests and IQ test for measuring cognitive ability, e.g. attention and concentration, the ability to learn complex tasks and concepts, memory, information processing, visuospatial function, the ability to produce and understanding language, the ability to solve problems and make decisions, and the ability to perform executive functions; for example, the General Practitioner Assessment of Cognition (GPCOG) test, the Memory Impairment Screen, the Mini Mental State Examination (MMSE), the California Verbal Learning Test, Second Edition, Short Form, for memory, the Delis-Kaplan Executive Functioning System test, and the like. Examples of vision tests include, for example, visual acuity tests, fundoscopy, and the like.

The calculation of the effective amount or effective dose of agent to be administered is within the skill of one of ordinary skill in the art, and will be routine to those persons skilled in the art. Needless to say, the final amount to be administered will be dependent upon the route of administration and upon the nature of the disorder or condition that is to be treated.

The subject methods and compositions may be used in research, e.g. in in vitro screens to identify new therapies for the treatment of cognitive and visual diseases, e.g. Aβ-associated diseases of the central nervous system. As another example, the subject methods and compositions may be used in vivo for the treatment of Aβ-associated diseases and disorders, e.g. Alzheimer's Disease, CAA, Down's syndrome, amblyopia, and glaucoma.

Cells may be from any mammalian species, e.g. murine, rodent, canine, feline, equine, bovine, ovine, primate, human, etc. For in vitro studies, cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

If the cells are primary cells, they may be harvest from an individual by any convenient method. For example, cells, e.g. blood cells, e.g. leukocytes, may be harvested by apheresis, leukocytapheresis, density gradient separation, etc. As another example, cells from solid tissues, e.g. neurons, may be harvested by biopsy. An appropriate solution may be used for dispersion or suspension of the harvested cells. Such solution will generally be a balanced salt solution, e.g. normal saline, PBS, Hank's balanced salt solution, Iscoves, etc., conveniently supplemented with fetal calf serum and/or other factors, e.g. B27, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

In practicing the subject methods, the subject composition may be provided to the cells one or more times, e.g. one time, twice, three times, or more than three times, and the cells allowed to incubate with the subject agent for some amount of time following each contacting event e.g. 16-24 hours, after which time the media is replaced with fresh media and the cells are cultured further.

In cases in which two or more different subject agents are provided to the cell, i.e. a cocktail of agents, the agents may be provided simultaneously, e.g. as two polypeptides delivered simultaneously, as two nucleic acid vectors delivered simultaneously, or as a single nucleic acid vector comprising the coding sequences for both fusion polypeptides. Alternatively, they may be provided consecutively, e.g. the first subject agent being provided first, followed by the second subject agent, etc. or vice versa.

Contacting the cells with the subject agent in vitro may occur in any culture media and under any culture conditions that promote the survival of the cells. For example, cells may be suspended in any appropriate nutrient medium that is convenient, such as Iscove's modified DMEM or RPMI 1640, supplemented with serum, e.g. fetal calf serum, heat inactivated goat serum (about 5-10%) etc., or synthetic reagents that support growth, e.g. B27, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Examples of mediums and reagents that find particular use in the culturing of neurons may be found in the Example section below.

As discussed above, the subject methods and compositions find use in reducing the effects on cells in vivo as well. In these in vivo embodiments, the subject agent is administered directly to the individual. Any mammal may be administered with the subject agent e.g. murine, rodent, canine, feline, equine, bovine, ovine, primate, human, etc. The subject agent may be administered by any of a number of well-known methods for the administration of polypeptides, peptides, small molecules or nucleic acids to a subject, e.g. as described herein or known in the art.

In some embodiments, the composition may be provided in conjunction with a second agent that modulates synapse maintenance and/or synaptic plasticity. In other words, an agent that reduces synapse loss or loss of synaptic plasticity, or that promotes synapse formation and/or promotes synaptic plasticity, may be provided in combination with the presently described agent. Any convenient second agent that modulates synapse maintenance and/or synaptic plasticity may be employed. For example, the agent may inhibit the activation of the complement pathway. Complement is a system of plasma proteins that interacts with the cell surfaces of pathogens or cells to mark them for destruction by phagocytes. The complement pathway has been implicated in promoting synapse loss and cognitive or visual decline in diseases such as Alzheimer's Disease and glaucoma, as well as in the cognitive decline associated with aging. Examples of therapeutic agents that inhibit complement signaling are provided in, for example, U.S. application Ser. No. 13/586,556, the full disclosure of which is incorporated herein by reference.

In some embodiments, the composition may be provided in conjunction with a second agent that has been demonstrated in the art to modulate Aβ oligomer effects on a cell. For example, an agent that reduces the amount of Aβ peptide produced, or that reduces the aggregation of Aβ peptides into oligomers, or that promotes the clearance of Aβ oligomers from the CNS, may be provided in combination with the presently described agent that inhibits the effects of Aβ oligomers on cells. Examples of such agents include β-secretase inhibitors, which block the first cleavage of APP outside of the cell; γ-Secretase inhibitors (e. g. semagacestat), which block the second cleavage of APP in the cell membrane to stop the subsequent formation of Aβ and its toxic fragments; selective Aβ42 lowering agents (e. g. tarenflurbil), which modulate γ-secretase to reduce Aβ42 production in favor of other (shorter) Aβ versions; immunotherapeutics which stimulate the host immune system to recognize and attack Aβ; and antibodies that either prevent plaque deposition or enhance clearance of plaques or Aβ oligomers. One such beta-amyloid vaccine that is currently in clinical trials is CAD106; and anti-aggregation agents such as apomorphine, which prevent Aβ fragments from aggregating or clear aggregates once they are formed.

In some embodiments, the composition may be provided in conjunction with a second agent that has been demonstrated in the art to treat a neurodegenerative disease or cognitive impairment. For example, a number of agents have been shown to have some efficacy in treating the cognitive symptoms of Alzheimer's disease (e.g., memory loss, confusion, and problems with thinking and reasoning), e.g. cholinesterase inhibitors (e.g. Donepezil, Rivastigmine, Galantamine, Tacrine), Memantine, and Vitamin E. As another example, a number of agents have been shown to have some efficacy in treating behavioral or psychiatric symptoms of Alzheimer's Disease, e.g. citalopram (Celexa), fluoxetine (Prozac), paroxeine (Paxil), sertraline (Zoloft), trazodone (Desyrel), lorazepam (Ativan), oxazepam (Serax), aripiprazole (Abilify), clozapine (Clozaril), haloperidol (Haldol), olanzapine (Zyprexa), quetiapine (Seroquel), risperidone (Risperdal), and ziprasidone (Geodon).

In some embodiments, the subject composition is provided before the second agent. In some embodiments, the subject composition is provided after the second agent. In some embodiments, the subject composition is provided concurrently with the second agent. In certain such embodiments, the subject composition comprises one or more of these additional agents.

In some aspects of the subject methods, the method further comprises the step of identify an individual in need of treatment by the subject methods, e.g diagnosing an individual as having a cognitive or visual impairment, diagnosing an individual as having an Aβ-associated disease or disorder, etc. Methods for measuring cognitive or visual function and identifying an individual having a cognitive or visual impairment are well known in the art, any of which may be used to identify an individual in need of treatment by the subject methods. For example, measuring a cognitive impairment may include administering a standardized learning task or IQ test, and comparing the results of the task/test with a reference, e.g. the results of the test at an earlier time in the individual's life, or the results of the test from a healthy, i.e. non-affected, individual. Cognition tests and IQ test for measuring cognitive ability and cognitive impairment, e.g. attention and concentration, the ability to learn complex tasks and concepts, memory, information processing, visuospatial function, the ability to produce and understanding language, the ability to solve problems and make decisions, and the ability to perform executive functions, are well known in the art, and include, for example, the General Practitioner Assessment of Cognition (GPCOG) test, the Memory Impairment Screen, the Mini Mental State Examination (MMSE), the California Verbal Learning Test, Second Edition, Short Form, for memory, the Delis-Kaplan Executive Functioning System test, and the like. As another example, measuring a visual impairment may include administering a visual acuity test, and comparing the results of the test with a reference, e.g. the results of the test at an earlier time in the individual's life, or the results of the test from a healthy, i.e. non-affected, individual.

In some aspects of the subject methods, the method further comprises the step of measuring cognitive ability, vision, synaptic plasticity, etc. after treatment, e.g. using the methods described herein or known in the art; and detecting a decreased rate of cognitive decline/visual decline/loss of synaptic plasticity, a stabilization of cognitive ability/visual ability/synaptic plasticity, and/or an increase in cognitive ability/visual ability/synaptic plasticity after administration of the subject compositions as compared to the cognitive ability/visual ability/synaptic plasticity of the individual before the subject composition was administered. In some instances, the determination is made by comparing the results of the cognition test, vision test or synaptic plasticity test to the results of the test performed on the same individual at an earlier time, e.g. 1 week earlier, 2 weeks earlier, 1 month earlier, 2 months earlier, 3 months earlier, 6 months earlier, 9 months earlier, 1 year earlier, 2 years earlier, 5 years earlier, or 10 years earlier, or more. In other instances, the determination is made by comparing the results of the cognition test, vision test, or synaptic plasticity test to the results of the test performed on a reference individual, e.g. a healthy individual that does not suffer from any greater cognitive or visual impairment than that associated with the natural aging process (a negative control), or, e.g. an individual that does suffer from Aβ-associated cognitive impairment.

### Utility

The subject methods and compositions find a number of uses in research and medicine. For example, the subject methods and compositions may be used to treat, treat, i.e. slow, halt, and in some instance reverse, the cognitive or visual decline that is a symptom of Aβ-associated diseases and disorders of the nervous system, e.g. the central nervous system, and in improving cognition and vision in Aβ-associated CNS disorders associated with impaired cognitive or visual function.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease. The terms “individual,” “subject,” “host,” and “patient,” used interchangeably herein, refer to a mammal, including, but not limited to, murines, simians, humans, mammalian farm animals, mammalian sport animals, and mammalian pets.

By “cognition” it is meant the mental processes that include attention and concentration, learning complex tasks and concepts, memory (acquiring, retaining, and retrieving new information in the short and/or long term), information processing (dealing with information gathered by the five senses), visuospatial function (visual perception, depth perception, using mental imagery, copying drawings, constructing objects or shapes), producing and understanding language, verbal fluency (word-finding), solving problems, making decisions, and executive functions (planning and prioritizing). Cognition is a faculty for the processing of information, applying knowledge, and changing preferences. By “cognitive plasticity” it is meant the ability to learn, e.g., the ability to learn complex tasks and concepts, analogous to the ability to learn of an organism that is undifferentiated such as a newborn or juvenile, e.g., a human from the time of birth to pre-pubertal age of about 10 years. By “cognitive decline”, it is meant a progressive decrease in cognition, as evidenced by, for example, a decline in one or more of, e.g., attention and concentration, learning complex tasks and concepts, memory (acquiring, retaining, and retrieving new information in the short and/or long term), information processing (dealing with information gathered by the five senses), visuospatial function (visual perception, depth perception, using mental imagery, copying drawings, constructing objects or shapes), producing and understanding language, verbal fluency (word-finding), solving problems, making decisions, and executive functions (planning and prioritizing). By “an impairment in cognitive ability”, “reduced cognitive function”, and “cognitive impairment”, it is meant a reduction in cognitive ability relative to a healthy individual, e.g. an age-matched healthy individual, or relative to the ability of the individual at an earlier point in time, e.g. 2 weeks, 1 month, 2 months, 3 months, 6 months, 1 year, 2 years, 5 years, or 10 years or more previously.

In some instances, treatment by methods of the present disclosure slows, or reduces, the progression of cognitive decline. In other words, cognitive abilities in the individual decline more slowly following treatment by the disclosed methods than prior to or in the absence of treatment by the disclosed methods. In some instances, treatment by methods of the present disclosure stabilizes the cognitive abilities of an individual. For example, the progression of cognitive decline in an individual is halted following treatment by the disclosed methods. In other words, no (further) cognitive impairment is observed. In some instances, treatment by methods of the present disclosure reduces, or reverses, cognitive impairment, e.g. as observed by improving cognitive abilities in an individual suffering from cognitive decline. In other words, the cognitive abilities of the individual following treatment by the disclosed methods are better than prior to treatment by the disclosed methods, i.e. they improve upon treatment. In some instances, treatment by methods of the present disclosure abrogates cognitive impairment. In other words, the cognitive abilities of the individual are restored, e.g. to a level observed of the individual when the individual was about 40 years old or less, following treatment by the disclosed methods, e.g. as evidenced by improved cognitive abilities in an individual.

Methods for measuring cognitive function are well known in the art, any of which may be used to identify an individual in need of treatment by the subject methods and/or to measure the cognitive stabilization or improvement in an individual during/after treatment with the subject methods. These include, for example, administering a standardized learning task or IQ test to the individual, and comparing the results of the task/test with a reference. In some instances, the reference may be the results of the task/test performed by one or more age-matched individuals that either experience reduced cognitive function (i.e. positive controls) or do not experience reduced cognitive function (i.e. negative controls). In some instances, the reference may be the results of the task/test performed by the same individual at an earlier age, e.g. 1 week earlier, 1 month earlier, 3 months earlier, 6 months earlier, 9 months earlier, and the like, for example to determine if the individual is suffering from cognitive decline.

### Screening Methods

The methods described above provide a useful system for screening candidate agents for the ability to inhibit synapse loss, loss of synaptic plasticity, and the associated cognitive or visual decline. For example, screening for candidate agents that prevent Aβ-induced loss of synapses and loss of synaptic plasticity in neurons that express PirB/LILRB2 should identify agents that will be useful in protecting those neurons from the effects of Aβ oligomers in vivo, which, in turn, will reduce, halt or even reverse cognitive or visual decline and reduced cognitive or visual function in patients with Aβ-associated conditions relative to untreated patients.

For example, in screening assays for biologically active agents, cells expressing PirB or LILRB2 are contacted with Aβ oligomers and a candidate agent of interest, and the effect of the candidate agent on modulating synapse numbers and synaptic plasticity is assessed by monitoring one or more output parameters. Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. Thus, for example, one such method may comprise contacting a cell that expresses PirB or LILRB2 with the Aβ oligomers and a candidate agent; and comparing the parameter to the parameter in a cell that expresses the PirB or LILRB2 and that was contacted with the Aβ oligomers but was not contacted with the candidate agent, wherein a difference in the parameter in the cell contacted with the candidate agent indicates that the candidate agent will reduce cell sensitivity to the Aβ oligomers.

One example of an output parameter that may be quantified when screening for, e.g., agents that modulate cellular sensitivity to Aβ oligomers would be to assess the neurons for their ability to undergo long term potentiation (LTP) or long term depression (LTD). Other examples would include assessing the number of dendritic spines, the amount of neurotransmitter transcribed and translated after a given amount of time exposed to agent, or the number of neurotransmitter vesicles present at the synapse. For example, for synapse quantification, cultures would be fixed, blocked and washed, then stained with antibodies specific synaptic proteins, e.g. synaptotagmin, PSD-95, etc. and visualized with an appropriate reagent, as known in the art. Analysis of the staining may be performed microscopically. In one embodiment, digital images of the fluorescence emission are with a camera and image capture software, adjusted to remove unused portions of the pixel value range and the used pixel values adjusted to utilize the entire pixel value range. Corresponding channel images may be merged to create a color (RGB) image containing the two single-channel images as individual color channels. Co-localized puncta can be identified using a rolling ball background subtraction algorithm to remove low-frequency background from each image channel. Number, mean area, mean minimum and maximum pixel intensities, and mean pixel intensities for all synaptotagmin, PSD-95, and colocalized puncta in the image are recorded and saved to disk for analysis. Yet other output parameters could include the activation state of the PirB/LILRB2 protein, e.g. the extent of phosphorylation observed on the ITIM domains of the PirB/LILRB2 cytoplasmic tail, the extent of cofilin activation, or the extent to which actin fibers become disassembled in the presence of Aβ and the candidate agent, e.g. as when non-neuronal cells are employed. In some instances, one parameter is measured. In some instances, multiple parameters are measured.

Cells useful for screening include any cell that expresses PirB or LILRB2 on its surface. For example, the cell may be a neuron, e.g. retinal ganglion cells (RGCs), cortical projection neurons, etc. In one embodiment of the invention, the neurons are a primary culture, e.g. of RGCs or cortical neurons. The cells are cultured in suitable medium, which will usually comprise appropriate growth factors, e.g. CNTF; BDNF; etc. The neurons are cultured for a period of time sufficient allow robust process outgrowth and then cultured with a candidate agent for a period of about 1 day to 1 week. In some embodiments, the neurons are cultured on a live astrocyte cell feeder in order to induce signaling for synapse formation. Methods of culturing astrocyte feeder layers are known in the art. For example, cortical glia can be plated in a medium that does not allow neurons to survive, with removal of non-adherent cells. In other embodiments, the cell may be non-neural, e.g. a fibroblast, a cell line, etc.

In other examples of screening assays for biologically active agents, the screen is a cell-free system. For example, candidate agents may be screened for their ability to interfere with binding to a PirB/LILRB2 extracellular domain (ECD) polypeptide by, e.g. surface plasmon resonance. In one such design, PirB/LILRB2 ECD polypeptide is immobilized to sensor surface, Aβ oligomer and candidate agent are passed over the sensor surface, and the binding of the Aβ oligomer in the presence of candidate agent is detected as a change in refractive index at the interface between the surface and a solution flowing over the surface changes. Alternatively, Aβ oligomer may be immobilized to the sensor surface, PirB/LILRB2 ECD polypeptide and candidate agent may be passed over the sensor surface, and the binding of the PirB/LILRB2 ECD polypeptide and candidate agent may be detected. A change in the refractive index in the presence of candidate agent relative to in the absence of candidate agent indicates that the agent has an effect on binding of Aβ oligomer to PirB/LILRB2.

Candidate agents of interest for screening include known and unknown compounds that encompass numerous chemical classes, primarily organic molecules, which may include organometallic molecules, inorganic molecules, genetic sequences, etc. as described above. An important aspect of the invention is to evaluate candidate drugs, including toxicity testing; and the like. Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. Methods of providing these vectors are well known in the art. Candidate agents of interest for screening also include polypeptides, e.g. antibodies. Candidate agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of cell samples, usually in conjunction with cells not contacted with the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

Inhibiting PirB Increases Neural Plasticity and Facilitates Recovery from Amblyopia in Visual Cortex.

A limited number of candidate molecules that appear to act as endogenous negative regulators of cortical plasticity have been identified. One such molecule, Paired Immunoglobulin-like Receptor B (PirB), is expressed in cortical and hippocampal neurons as well as in some immune cells. In the nervous system, PirB binds several ligands, including Major Histocompatibility Class I proteins, NogoA, and myelin components. Both in immune cells and neurons, ligand binding recruits SHP-1 and -2 phosphatases. SHP recruitment requires PirB phosphorylation on its ITIM (Immunoreceptor Tyrosine-based Inhibitory Motif) domains. In neurons, cofilin is also recruited to PirB, leading to changes in the actin cytoskeleton. Germline PirB−/− mice have enhanced OD plasticity not only during the critical period but also beyond, and they recover more rapidly in a stroke model. PirB, and its human ortholog Leukocyte immunoglobulin-like receptor, subfamily B, member 2 (LilrB2), bind soluble amyloid-β oligomers, and germ-line PirB deletion rescues OD plasticity and hippocampal deficits in a mouse model of Alzheimer's disease. However, it remains unknown whether the enhanced OD plasticity and stroke recovery in germline PirB^{-/-} mice are due to early developmental changes, or whether PirB acts at all ages to limit plasticity, which would make it an attractive therapeutic target for drug development. Since PirB is a receptor, signaling can be modulated by conditional genetic knockout or by interfering with ligand binding. If PirB functions throughout life, disrupting PirB should enhance plasticity or facilitate recovery at any age.

### Results

Genetic Deletion of PirB Enhances OD Plasticity.

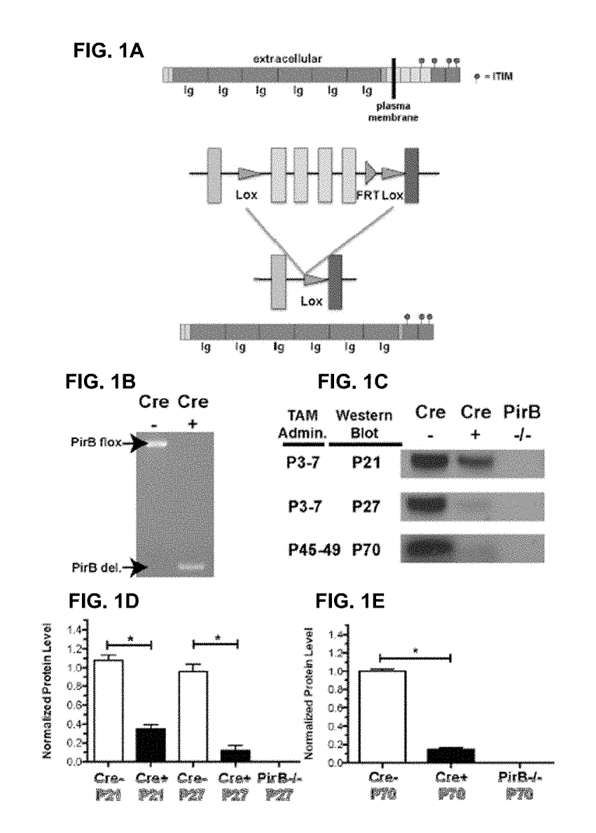

To disrupt PirB function with temporal control, a conditional allele of PirB was generated by inserting loxP sites surrounding exons 10-13, which contain the transmembrane domain and first ITIM domain of PirB (FIG. 1A). To obtain robust widespread deletion, this PirB flox mouse line was crossed with a transgenic mouse line expressing tamoxifen-inducible Cre-ERT2 on a ubiquitin C promoter. The resulting Ubc-CreER_{T2}; PirB flox/flox mice were bred with PirB flox/flox mice, producing experimental Ubc-CreERT2; PirB flox/flox animals (henceforth called Cre+) as well as PirB flox/flox (Cre−) littermate controls. Tamoxifen injections given either neonatally or after critical period closure induced robust deletion of the floxed allele from genomic DNA within one week (FIG. 1B).

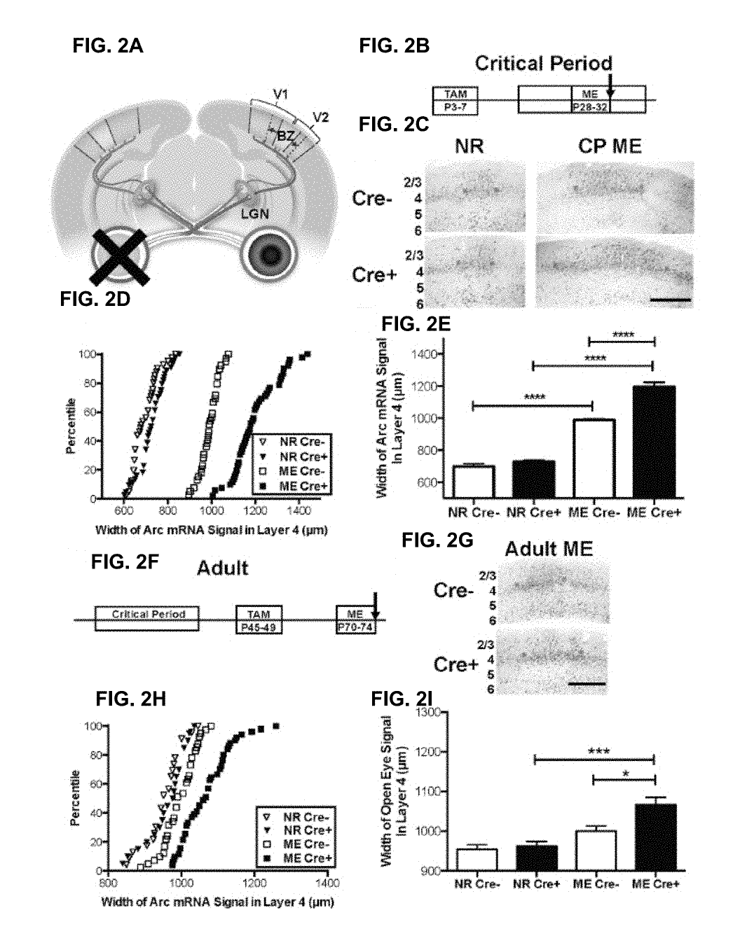

PirB protein loss was more gradual; for example, daily tamoxifen treatment from P3 to P7 diminished PirB protein in the forebrain by ˜90% by P27 (FIG. 1C, D). A similar gradual loss of protein was seen at P70 after tamoxifen treatment from P45 to P49 (FIG. 1C, E). Thus tamoxifen administration substantially reduced PirB protein levels by the peak of the OD critical period at P28, as well as in adulthood by P70. To assess whether OD plasticity during the critical period is increased by acute postnatal removal of PirB, mice received ME from P28 to P32. At P32, we used the method of Arc mRNA induction to assess how much the functional representation of the spared eye had expanded within visual cortex. Arc is an immediate early gene induced within minutes of visual stimulation, and the upregulated mRNA can be detected in cortical neurons functionally driven by the stimulated eye. We measured the horizontal extent of the Arc mRNA in situ hybridization signal along L4 of visual cortex ipsilateral to the spared, stimulated eye (FIG. 2A-C). This expansion in width of Arc mRNA signal is a reliable measure of open eye strengthening after visual deprivation and correlates well with other methods used to assess OD plasticity including single unit electrophysiology, visual evoked potentials or intrinsic signal imaging. The width of Arc mRNA induction does not expand in transgenic mice known to lack OD plasticity as measured by other methods, while there is an increase in width of Arc mRNA signal in mice known to have increased OD plasticity.

As expected during the critical period, 4 days of ME in either Cre⁺ or Cre− mice caused substantial expansion in width of Arc mRNA signal as compared to normally reared controls (FIG. 2B-E). However, in mice lacking PirB (Cre+), the spared-eye representation expanded 21% more than control (Cre−) littermates, while in normally reared mice, there was no difference between genotypes in the width of Arc mRNA signal induced by stimulation of the ipsilateral eye (FIG. 2E). A two-way ANOVA confirmed a significant interaction effect between visual manipulation and genotype (p<0.0001). To further facilitate comparisons between genotypes, a plasticity index was calculated by normalizing the width of Arc mRNA induction following ME to the normally reared value for each genotype. The plasticity index in Cre+ mice is 23% higher than in Cre− mice, consistent with the absence of PirB. These observations imply that PirB does not act only early in fetal life, but rather actively represses OD plasticity during the critical period.

In developing WT animals, OD plasticity decreases between P35 to P40 (the end of the critical period), and short periods of MD or ME (3-4 days) thereafter have little effect on ocular dominance. To determine whether decreasing PirB function might enhance plasticity in adulthood, tamoxifen was administered from P45 to P49 (FIG. 2F), which resulted in an almost complete loss of PirB protein by P70 (FIG. 1C, E). Then these adult mice received ME from P70 to 74, weeks after the critical period has normally closed. As with juvenile mice, at P74 there was no significant difference in baseline width of Arc mRNA signal between genotypes in normally reared controls (FIG. 2G-I). There was also no significant difference in width of Arc mRNA signal following ME from P70 to P74 in Cre− controls, as expected given the brief 4-day period of deprivation at this older age. However, we observed a significant expansion of open-eye representation (p=0.0004) in Cre− mice (which lacked PirB) with 4 days of ME (FIG. 2H, I). In adults, as in juveniles, a two-way ANOVA confirmed that there was a significant interaction between visual manipulation and genotype (p=0.04). Although the magnitude of adult OD plasticity was reduced in all genotypes as compared to that observed during the critical period, deletion of PirB in adults was sufficient to enhance OD plasticity and to cause a significant expansion in open eye representation not observed in adult wild type littermate controls with normal PirB levels.

Postnatal Deletion of PirB from Excitatory Neurons Enhances Adult OD Plasticity.

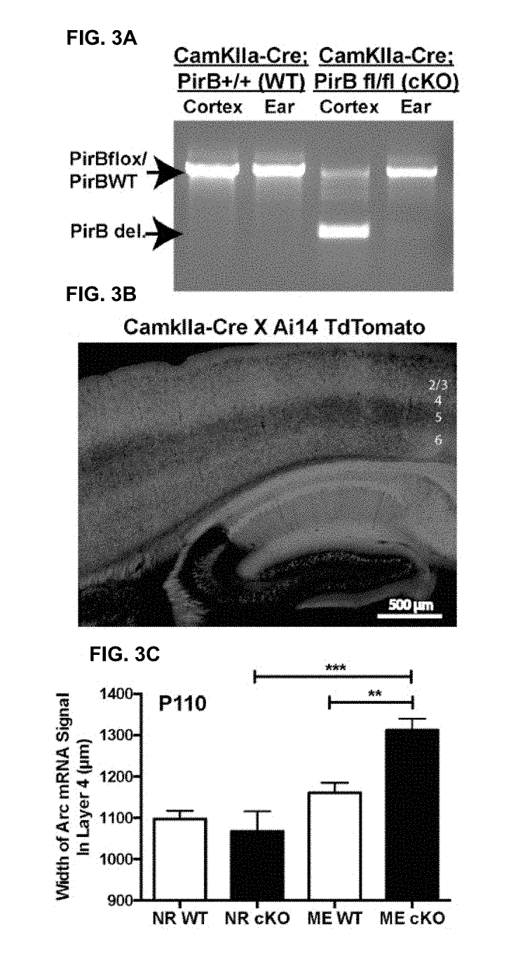

In the experiments above, a ubiquitously expressed Cre recombinase was used to achieve robust deletion of PirB in all cells. Next, we investigated whether loss of PirB specifically in forebrain excitatory neurons was sufficient to enhance OD plasticity. PirB flox/flox mice were crossed with a CamKIIa-Cre line, which expresses Cre exclusively in forebrain excitatory neurons, generating CamKIIa-Cre; PirB flox/flox conditional knockouts, and CamKIIa-Cre; PirB+/+ littermate controls. PCR genotyping of brain and ear confirms brain-specific deletion of the floxed region of PirB (FIG. 3A). To confirm the spatial pattern of Cre deletion, CamKIIa-Cre mice were also crossed to the Ai14 TdTomato reporter line. Results show faithful Cre activity at P30 in pyramidal neurons of hippocampus and cortex (FIG. 3B). Prior studies have shown that excision of floxed regions of DNA in this Cre line is gradual, with complete deletion occurring around 3 months of age, permitting us to examine effects of PirB deletion in adulthood.

CamKIIa-Cre; PirB flox/flox conditional knockouts, and CamKIIa-Cre; PirB^{+/+} littermate controls received 10 days of ME from P100 to P110, followed by assessment of OD plasticity. Open eye expansion in visual cortex in CamKIIa conditional knockout mice was ˜13% greater than in littermate controls (FIG. 3C), suggesting that postnatal deletion of PirB from excitatory neurons is sufficient to increase OD plasticity by P110. A two-way ANOVA confirmed significant interaction between visual manipulation and genotype (p=0.007). Furthermore, this increase is similar in magnitude to prior observations of enhanced OD plasticity in adult mice with PirB germline deletion, implying that loss of PirB function in excitatory neurons may be largely responsible for the observed PirB−/− phenotype.

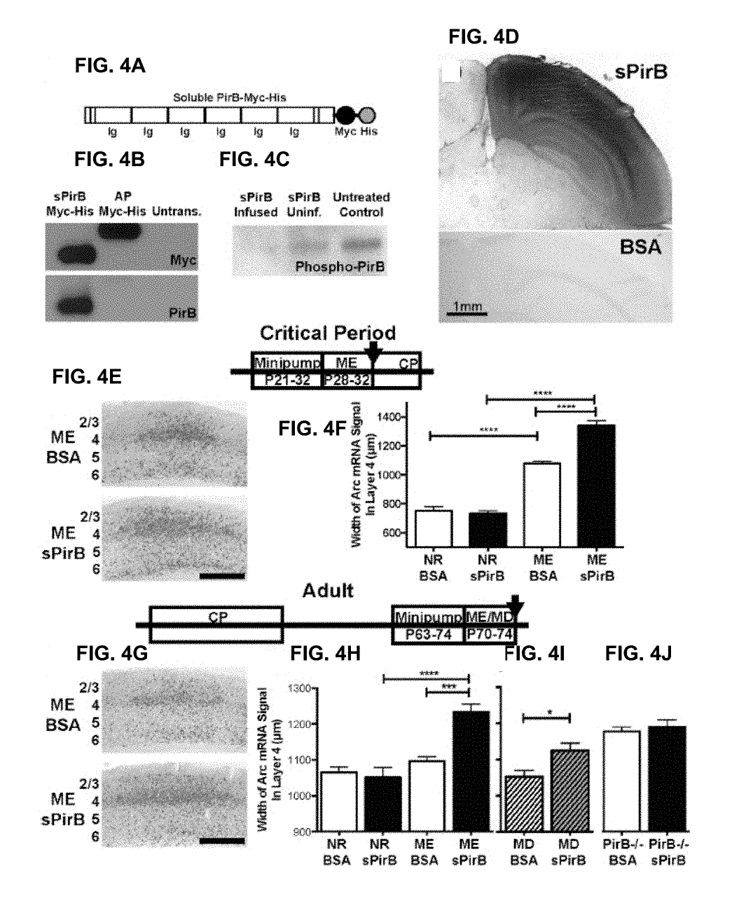

Rapid enhancement of OD plasticity by blockade of PirB ligand binding. The genetic approaches used above excise PirB from the genome postnatally, but the ensuing loss of PirB protein is gradual and widespread. These experiments establish that PirB actively functions throughout the animals' lives. Next, a molecular-pharmacological approach was employed as a proof-of-concept for a therapeutic reagent that blocks PirB function, and to achieve rapid and local disruption of PirB function within visual cortex. We generated a soluble PirB ectodomain (sPirB) protein, containing the first 6 Ig-like domains and His and Myc tags for purification and detection (FIG. 4A). Soluble ectodomains of receptors act as “decoys” to sequester endogenous ligands, thus reducing ligand binding and subsequent receptor signaling. They have been used frequently in experimental contexts, and several are currently on the market as therapeutics. Previous studies have demonstrated that soluble versions of the full PirB ectodomain with similar sequence to the one used here can bind known PirB ligands including MHC Class I, myelin components NogoA, OMgp, and MAG, and amyloid β oligomers. However the PirB ectodomain has never been used therapeutically. sPirB blocks PirB binding to endogenous ligands and therefore reduce downstream signaling.

We transfected a plasmid coding for sPirB into HEK293 cells, and the recombinant protein was then secreted into culture supernatant. We detected sPirB by western blotting against both the Myc tag and the PirB ectodomain (FIG. 4B). sPirB was then produced at large scale and purified via Ni-His column. Next, either sPirB or Bovine Serum Albumin (BSA) was infused into visual cortex (V1) of WT mice during the critical period via osmotic minipumps. To assess efficacy of sPirB infusions, minipumps were implanted at P21. At P28 cortical tissue was harvested posterior to the implantation site in the infused and contralateral hemispheres, as well as in uninfused littermates. PirB phosphorylation was decreased in visual cortex posterior to the infusion site compared both to the contralateral hemisphere and to untreated littermate controls (FIG. 4C). Eleven days after implantation, extensive diffusion of sPirB can be detected by anti-Myc immunostaining of sections (FIG. 4D) across visual cortex as far as 2-mm posterior to the infusion site (Figure S3B); no anti-Myc staining was detected in BSAinfused control brains.

Minipump infusion of sPirB into visual cortex of WT mice for 11 days during the critical period (P21 to P32), combined with ME from P28 to P32, resulted in a pronounced expansion in width of visual cortex containing neurons functionally driven by the open (ipsilateral) eye, as assessed with Arc mRNA induction (FIG. 4E). By this measure, OD plasticity was 24% greater with sPirB infusion than in controls infused with an equivalent concentration of BSA (FIG. 4F); a two-way ANOVA confirmed significant interaction between visual manipulation and treatment (p=0.0001). Because the infusion was local and limited to an 11 day period beginning at the onset of the critical period, the results of this experiment help to narrow considerably the spatiotemporal window in which PirB acts to suppress plasticity. Taken together with the tamoxifen-inducible PirB knockout results presented above, our data suggest that PirB actively suppresses plasticity locally in visual cortex during the critical period for OD plasticity.

Strikingly, substantial OD plasticity could be restored to the visual cortex of adult WT mice after minipump infusions of sPirB from P63 to 74. ME from P70 to 74 resulted in significant expansion of the functional representation of the open eye in the presence of sPirB, but not BSA, (FIG. 4G, H). This effect of sPirB was twice as large as that of tamoxifen-driven PirB excision in adult mice (FIG. 2I). In contrast, there was no significant effect of 11-day minipump infusions of either BSA or sPirB on the width of Arc mRNA induction following stimulation of the ipsilateral eye in mice reared with normal vision (FIG. 4H: NR sPirB). Consistent with the idea that sPirB affects the expansion rather than the baseline width of Arc induction, a two-way ANOVA detects a significant interaction effect between visual deprivation and treatment (p=0.001).

We further assessed the effect of sPirB on adult OD plasticity using monocular deprivation (MD) (as opposed to monocular enucleation). In adult mice, a brief period of MD such as 3 days does not usually produce a shift in ocular dominance favoring the open eye (FIG. 4H, I). Therefore, we examined whether MD from P70 to 73, coupled with sPirB treatment, would result in open eye expansion. Similar to results with ME, sPirB-treated adult mice receiving MD had a significant increase in open eye expansion, with no change in BSA-treated controls (FIG. 4I). Therefore, in adult visual cortex sPirB treatment during brief periods of either MD or ME generates detectable OD plasticity.

PirB binds multiple ligands, which themselves can bind to other receptors, possibly accounting for the observation that sPirB has a larger effect on OD plasticity than genetically induced deletion of PirB. To test this idea, we infused sPirB or BSA by minipumps into adult germ-line PirB−/− mice, which also received 4 day ME (P70 to P74). Germline PirB^{-/-} mice implanted with BSA minipumps demonstrated the expected increase in OD plasticity when compared to WT mice receiving BSA (FIG. 4H, 4J). However, there was no additional effect of sPirB infusion compared to BSA infusion in PirB−/− mice (FIG. 4J). This lack of effect of sPirB infusion in adult PirB−/− visual cortex suggests that endogenous PirB is required to enhance OD plasticity and that the pharmacological blockade is PirB-specific. Together with results from the tamoxifen-inducible PirB mice, these findings indicate that acute, specific manipulations that delete or block PirB function are sufficient to enhance OD plasticity even when administered well beyond the end of the critical period.

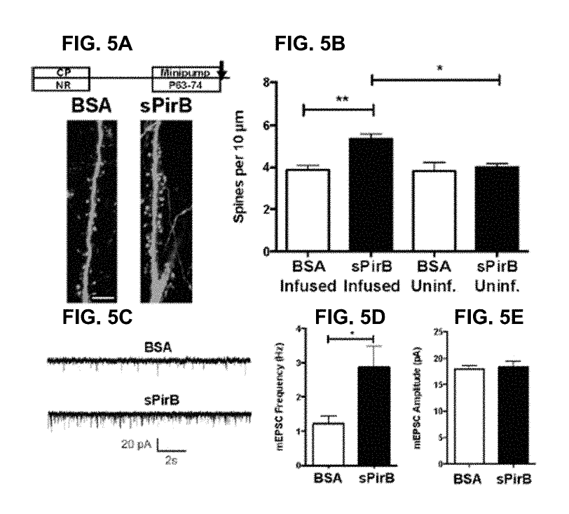

sPirB increases spine density and functional synapses on L5 pyramidal neurons. There is a significant increase in spine density on the apical tufts of L5 pyramidal neurons in visual cortex of germline PirB knock out mice reared with normal vision. It has been proposed that these extra spines represent a pre-existing structural substrate that is then recruited for the more rapid and robust OD plasticity observed in these mice. Indeed, prior sensory experience in visual or auditory systems increases plasticity accompanied by increased structural connectivity. We tested whether PirB might contribute to these structural changes. sPirB infusion might trigger an increase in spine density even without visual deprivation. To test this hypothesis, visual cortex of normally reared wild-type Thy-1 YFP-H transgenic mice, in which cortical L5 pyramidal neurons are YFP-labeled, received minipump infusions of either sPirB or BSA from P63 to 74 (FIG. 5A, B). Spines on apical dendrites of L5 pyramidal neurons were examined in the binocular zone at a distance posterior to the infusion site comparable to that studied above for assessment of OD plasticity. In this region after sPirB infusion, pyramidal neuron somata, dendrites and spines appeared intact and healthy, without fragmentation or blebbing (FIG. 5A). Spine density on L5 apical dendritic tufts of animals reared with normal vision was 38% greater in the presence of sPirB than of BSA (FIG. 5B). Spine density on L5 neurons in the uninfused hemisphere was not altered, and a two-way ANOVA confirms a significant interaction effect between hemisphere and treatment (p=0.03). The observed density increase could arise if sPirB acts on a subclass of dendritic spines. However after an 11 day infusion of either sPirB or BSA, there was no significant difference in the proportion of spines classified as mushroom, thin, or stubby. Together results show that in adult visual cortex it is possible to generate a local increase in spine density on L5 neurons by infusing sPirB, even in the absence of a visual manipulation or deprivation.

To examine if the increase in spine density represents new functional synapses, miniature excitatory postsynaptic currents (mEPSCs) were recorded from L5 pyramidal neurons in slices of visual cortex (P70 to P77), after 7 to 11 days of sPirB minipump infusion in vivo, in mice reared with normal binocular vision (FIG. 5C). mEPSC frequency was significantly greater following sPirB treatment than in BSA littermates (FIG. 5D), with no change in mEPSC amplitude (FIG. 5E). This finding is consistent that sPirB infusion causes an increase in synaptic connectivity, suggesting that newly spines formed represent sites of functional synapses.

sPirB treatment after long term monocular deprivation enables recovery of spine density. Long term monocular deprivation (LTMD) is a well-studied an animal model of amblyopia because it involves an experience-dependent developmental loss of function in the deprived eye. In rodents, LTMD profoundly decreases visual acuity, as well as the number of cortical neurons visually driven by the deprived eye. There is little if any recovery, even after restoration of binocular vision. It has been proposed that a decrease in dendritic spine density underlies these functional deficits. For example, LTMD generates a significant decline in spine density on basolateral dendrites of L5 pyramidal neurons contralateral to the deprived eye.

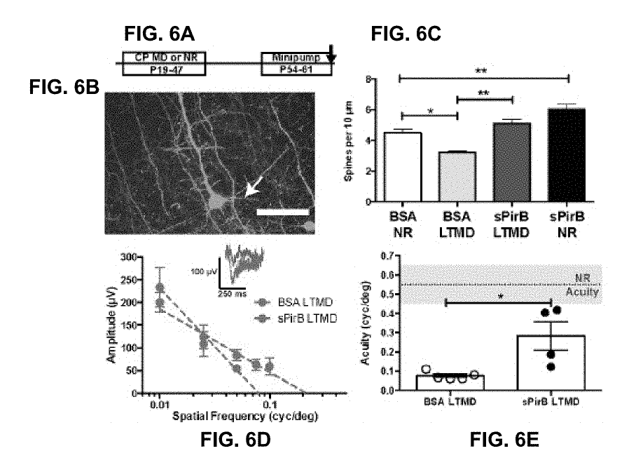

Given the rapid and generative effect of sPirB on spine density and mEPSC frequency described above in normal visual cortex, we wondered whether sPirB treatment might generate a spine density increase that could facilitate recovery from LTMD. Thy-1 YFP WT mice were either normally reared or received LTMD spanning the entire critical period for OD plasticity (P19 to 47). At P47, the deprived eye was reopened to restore binocular vision for 1 week. Then at P54, minipumps containing either sPirB or BSA were implanted in visual cortex contralateral to the deprived eye until P61 (FIG. 6A), at which time spine density on L5 basolateral dendrites was measured.

LTMD caused a 28% decrease in spine density along L5 basolateral dendrites in BSA treated controls despite two weeks of subsequent binocular vision (FIG. 6B, C), as expected from previous studies. In contrast, the spine loss accompanying LTMD was almost entirely reversed by minipump infusion of sPirB. There was a 57% greater basolateral dendritic spine density in sPirB-treated animals than LTMD BSA-treated controls, essentially restoring spine density to levels of BSA treated controls reared with normal vision (FIG. 6C). There was no detectable change in overall distribution of basolateral spine types, similar to observations above for L5 apical dendrites. In addition, in normally reared littermates infused with sPirB, spine density increased on basolateral dendrites compared to normally reared BSA infused controls, demonstrating that sPirB infusion can increase spine density not only on apical tufts of L5 pyramidal neurons (FIG. 5B), but also on their basolateral dendrites (FIG. 6C). Together these results demonstrate that sPirB treatment just for 1 week can reverse spine loss resulting from LTMD even when infused after a delay following eye reopening.

sPirB Induces Recovery of Visual Acuity after Long Term Monocular Deprivation

To assess whether the recovery in spine density could mediate recovery of visual function, visual evoked potential (VEP) recordings were made from cortical layer 4/5 to measure spatial frequency acuity. This method is well established, highly sensitive and tightly correlates with behavioral and single unit measurements of visual recovery after LTMD. Moreover, since the effects of sPirB minipump infusions are restricted to a local region of visual cortex (FIG. 5B), VEPs are well suited to assess recovery of deprived eye function specifically within the infused region. Visual acuity is measured by varying the spatial frequency of visual stimuli and monitoring the amplitude of VEP responses (FIG. 6D).

In normally reared Thy-1 YFP WT mice, visual acuity measurements were similar to previously reported values, approximately 0.55±0.1 cycles/degree (FIG. 6E, shaded region). After 4 weeks of LTMD from P19 to 47, BSA-treated control animals experienced a dramatic loss of visual acuity, with responses above noise level only to a maximum spatial frequency of 0.05 cyc/deg (FIG. 6D, E). This severe (85-90%) reduction in acuity (FIG. 6E) persisted for 2 weeks even after restoration of binocular vision, including 1 week of BSA minipump treatment. The lack of recovery is consistent with previous results suggesting that binocular vision alone is not sufficient for recovery of visual function after the critical period.

In contrast, there was significant improvement in visual acuity in mice receiving a 1-week minipump infusion of sPirB (FIG. 6D, E). Although the degree of recovery was variable among animals, 50% of the sPirB-treated mice recovered visual acuity to 0.4 cyc/degree or higher, nearly the normal range for unmanipulated mice (FIG. 6E). Even the remaining sPirB treated mice regained acuity higher than that measured in the highest LTMD BSA-treated controls. These results suggest that after just one week of sPirB treatment, there was significant functional recovery of visual acuity in the deprived eye, and that, in some cases, visual function rapidly recovered to nearly normal levels.

There are several major findings from this study. First, by acutely disrupting the function of the endogenous receptor PirB, we find that OD plasticity in visual cortex of adult mice can be enhanced long after critical period closure. Two independent but complementary methods were used—genetic deletion of PirB with temporal control, and blockade of ligand binding with a soluble PirB “decoy” protein. Both methods generated enhanced OD plasticity not only during the critical period but also in adulthood, phenocopying germline PirB knockouts. These observations suggest that endogenous PirB actively acts to repress cortical plasticity throughout life, validating PirB as a target for therapeutic interventions that could improve recovery from injury, correct dysfunctional developmental plasticity, or perhaps even temporarily enhance learning in normal individuals.

Second, we report that in a model of amblyopia it is possible to reverse both the spines and visual acuity loss in cortex after LTMD by restoring binocular vision coupled with an infusion of sPirB. Lastly, sPirB infusion into the visual cortex of normally reared mice produced rapid increases in spine density and functional synapses on L5 pyramidal neurons. Although motor learning, sensory enrichment, or brief MD all have been shown to produce rapid increases in spine density, sPirB treatment does so at a greater magnitude and in the absence of novel stimuli or training. Together our observations demonstrate that targeting and disrupting PirB function increases synaptic connectivity and plasticity even after the critical period. Since PirB is expressed by pyramidal neurons throughout the neocortex, our results may also apply to cortical areas beyond the visual system.

sPirB acutely regulates spine and synapse density. Infusion of sPirB decreases proximal PirB downstream signaling (FIG. 4C), consistent with its previously demonstrated sequestration of endogenous PirB ligands. In adulthood, acute blockade with sPirB results in enhanced OD plasticity and produces a rapid increase in spine density and mEPSC frequency, even in the absence of altered vision. Many interventions that affect synaptic connectivity and spine dynamics also enhance OD plasticity, including transplantation of inhibitory neuron progenitors or disruption of NgR1-NogoA function. Spine density increases also correlate with enhanced subsequent plasticity: studies have shown that new spines generated during an initial MD can be co-opted for more robust OD plasticity during a subsequent MD. Furthermore in germline PirB−/− mice, enhanced OD plasticity is associated with a large increase in spine density on L5 neurons, and an increase in the magnitude of L4 to L2/3 LTP in visual cortex. Collectively, these experiments connect an increase in spine density and functional connectivity to enhanced synaptic plasticity. Thus sPirB can generate greater OD plasticity by creating a more highly interconnected structural substrate that can be accessed for more rapid and robust synaptic change.

sPirB as a Therapy for Recovery from Amblyopia.

Long term monocular visual deprivation throughout the critical period, used here as an animal model of amblyopia, leads to a profound loss of visual acuity, as well as to loss of visual responsiveness of cortical neurons to stimulation of the deprived eye; both are highly resistant to recovery even when binocular vision is subsequently restored. Decreases in spine density on both L2/3 pyramidal cells and pyramidal neurons throughout cortex have also been reported following LTMD or chronic MD. Although reversal of this spine loss on cortical pyramidal neurons has been seen, reversal required suturing closed the formerly open eye in combination with fairly disruptive treatments such as chondroitinase ABC to digest extracellular matrix, or 10 days of dark exposure followed by visual stimulation. Recovery of spines in both of these cases was accompanied by robust recovery of VEP acuity. In our study, it was striking to find that sPirB infusion, combined with binocular vision, is sufficient by itself to bring spine density values and VEP acuity estimates close to normal. Visual acuity as measured by VEPs predicts physiologically relevant recovery of visual function in the deprived eye, indicating that vision through the deprived eye in sPirB treated mice has greatly improved. Together with the data on spine recovery, these results show that sPirB can enable significant structural and functional recovery from amblyopia after LTMD within just 7 days of treatment.

These observations indicate that sPirB—a soluble receptor ectodomain—is a therapeutic, and they provide proof-of-concept for generating other PirB blocking reagents. The standard treatment for human amblyopic patients mandates early intervention during a developmental critical period, and involves alternating patching between the two eyes to strengthen the amblyopic eye, but this treatment interferes with development of binocular depth perception. There are several PirB homologs in humans (LilrBs), and LilrB2 protein is expressed in the human brain. Targeting LilrB2 or other members of the LilrB receptor family might permit recovery from amblyopia without requiring eye patching, as implied by the results of the LTMD experiments in mice.

PirB:

An Endogenous Target for Manipulations of Synapse and Systems Level Plasticity. Our observations add to a growing body of research that has unmasked active roles for molecules in the brain acting as negative regulators of functional and structural plasticity both in development and in adulthood. In the case of PirB, this negative regulation may also be hijacked, as in Alzheimer's disease, where beta amyloid oligomers have been shown to bind to PirB/LilrB2 with nanomolar affinity, resulting in loss of OD plasticity and deficits in cortical and hippocampal synaptic plasticity. Thus, sPirB and human soluble receptor homologs may be viable therapeutics for AD. By generating a recombinant soluble PirB protein, we have demonstrated a utility for selectively blocking PirB receptor interaction with endogenous ligands. These results further support the value of creating PirB/LilrB antagonists that cross the blood-brain barrier, enhancing plasticity and increasing synapse and spine density in cases of disease, dysfunction, injury, or even for cognitive enhancement in normal individuals.

### Materials and Methods

Study Design: The objective of this study was to devise methods to delete PirB function acutely, then monitor the effects on measures of synaptic and OD plasticity, and recovery from LTMD. Two methods were used: tamoxifen-induced PirB deletion via a PirB conditional allele, or sPirB minipump infusion. Since OD plasticity is induced by changes in visual experience, experiments were designed to capture an interaction effect between genotype/treatment and visual manipulation; 4 groups and a two-way ANOVA design was used to test for interactions. All experiments were performed blind to genotype and/or treatment. Littermates were used to control for genetic variation, and mice were randomly assigned to different visual manipulations and treatments within a litter. To detect genotype effects similar or greater than those previously reported, sample sizes were chosen based on a statistical power of 80% with an alpha value of 0.05. The number of replicate measurements and animals are given in each figure legend.

Mouse strains: PirB−/− and PirB flox/flox mice were generated as described. A PirBWT line was maintained on the same background and used for all minipump infusion experiments performed during the critical period (P21 to 32). For adult minipump experiments (P63 to 74), PirBWT and PirB−/− mice were crossed with the Thy-1 YFP-H transgenic line (JAX #003782), which expresses YFP in a subset of L5 pyramidal neurons. For inducible knockout experiments, UbC-CreERT2 mice (JAX #007001) were bred with PirB flox mice to generate UbC-CreERT2; PirB flox/flox mice and PirB flox/flox littermates. For conditional knockout experiments, CamKIIa-Cre; PirB flox/+ mice were bred with PirB flox/+ mice to generate CamKIIa-Cre; PirB flox/flox mice and CamKIIa-Cre; littermates. CamKIIa-Cre mice were also bred with Ai14 TdTomato reporter mice. All experiments were performed in accordance with protocols approved by Stanford University Animal Care and Use Committee in keeping with the National Institutes of Health's Guide for the Care and Use of Laboratory Animals.

sPirB Protein Production: To create a soluble PirB mimic, the PirB ectodomain was cloned into a plasmid containing a His tag for purification and a Myc tag for antibody detection with a sequence identical to previous publications. For minipump infusions, Invitrogen Custom Services produced sPirB in larger quantities in FreeStyle HEK 293 cells and purified it on a nickel column (Ni-His, Invitrogen).

Osmotic Minipump implantations and sPirB infusion: Craniotomies were performed and minipumps (Alzet model 1002; 0.25 μL/hr, 100 μL capacity) containing either 1 mg/mL sPirB, or 1 mg/mL Bovine Serum Albumin (BSA, VWR EM-2930) in 0.1M phosphate buffered saline (PBS) were implanted subcutaneously, connected to a cannula. The cannula was inserted just anterior to primary visual cortex (2.5 mm lateral and 3 mm posterior to bregma).

Arc mRNA induction and in situ hybridization: Arc mRNA was induced by placing mice overnight in total darkness (16-18 hr), followed by bright illumination for 30 minutes to permit vision through the open eye prior to euthanasia via isoflurane anesthesia and decapitation. A digoxigenin-labeled Arc antisense mRNA probe was used for colorimetric in situ hybridizations performed on brain sections. Images were acquired via brightfield microscopy and analyzed using the Line Scan function of NeuroLens software to measure the width of the Arc mRNA hybridization signal ipsilateral to the open (nondeprived) eye along L4 of visual cortex, at the 3-4 border. Multiple sections were scanned and averaged per animal (e.g. FIG. 2).

Visual Evoked Potential Recordings: Animals were anesthetized with urethane (0.6-1.2 g/kg, Sigma) and chlorprothixene (5 mg/kg, Sigma), and at incisions with lidocaine (2%, Sparhawk Laboratories), then the scalp was exposed and the minipump cannula removed. Following a craniotomy centered over V1, a glass pipette filled with ACSF was inserted to record local field potentials at a depth of 450-600 μm. Responses to sinusoidal grating stimuli were averaged over stimulus blocks, and a peak response amplitude within a 500-ms window after stimulus onset was determined. Visual acuity was estimated by finding the x-intercept of a semi-logarithmic regression of response amplitudes across different spatial frequencies.

Statistical Analyses: All statistical analyses were performed with Prism software (Graphpad). When only two groups were involved, two-sample t-tests were used, with Welch's correction for unequal variances applied where appropriate. Data for which a normal distribution could not be assumed was analyzed with Mann-Whitney U tests. In cases where both treatment/genotype and visual manipulation or hemisphere were varied, a two-way ANOVA was conducted, with Tukey post-hoc tests for individual pairs of columns.

Arc mRNA Induction and In Situ Hybridization:

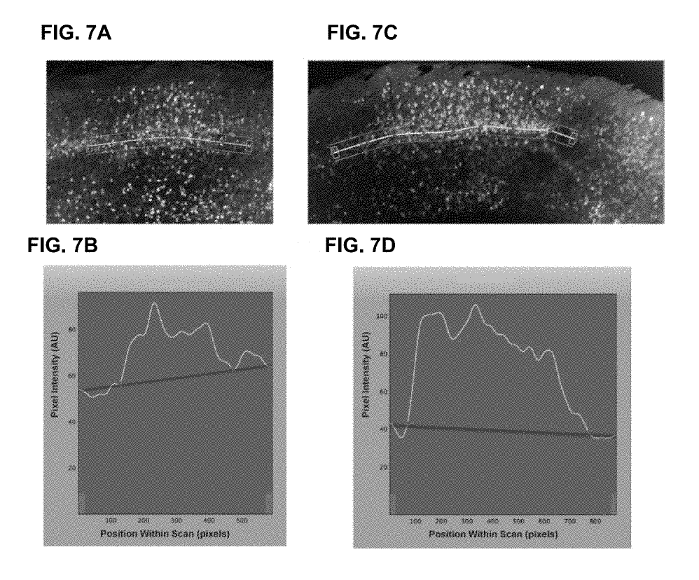

As described, Arc mRNA was induced by placing mice overnight in total darkness (16-18 hr), followed by bright illumination for 30 minutes to permit vision through the open eye prior to euthanasia via isoflurane anesthesia and decapitation. Brains were frozen and sectioned for subsequent in situ hybridization. A digoxigenin-labeled Arc antisense mRNA probe was generated and in situ hybridizations were performed as described previously. Probe was detected using a colorimetric visualization method. Images were acquired via brightfield microscopy, contrast-inverted to facilitate analysis, and analyzed using the Line Scan function of NeuroLens software to measure the width of the Arc mRNA hybridization signal ipsilateral to the open (nondeprived) eye along L4 of visual cortex, just at the L3-4 border (FIG. 7). This line scan was then combined with a linear background model, yielding a quantitative measure of the width of continuous cortical territory functionally activated by the ipsilateral eye. Approximately 6-8 sections containing visual cortex were scanned and averaged per animal, yielding one data point per individual. Data acquisition and analyses were all performed blind to genotype and/or treatment regime.

Tamoxifen Injections:

Tamoxifen free base (Sigma T5648) was dissolved at 20 mg/mL in a mixture of 2% ethanol in corn oil (Sigma C8267), and stored in aliquots at −20° C. For juvenile mice, 7 mg of tamoxifen was injected intraperitoneally into the mother daily when the pups were P3 to P7. For adult mice, 4 mg of tamoxifen were injected daily when mice were P45 to P49.

PCR Genotyping:

Samples from mouse cortex were lysed and genomic DNA extracted using the DNEasy Blood and Tissue Kit (Qiagen). Excision of PirB was detected using primers flanking the floxed region of PirB with sequences ctgccctcatgtcttaactt and gagaatcaccagacacatgc. Samples were run on a 1.5% agarose gel in TAE buffer, and visualized with ethidium bromide. Western Blotting for PirB: Mice were anesthetized with isoflurane, decapitated, and forebrains (cortex and hippocampus) were removed and placed in ice-cold lysis buffer (50 mM Tris, pH 7.4, 150 mM NaCl, 1% NP-40) with protease inhibitor cocktail (Sigma P8340-1ML), Samples were centrifuged to remove cellular debris, and then incubated overnight at 4° C. with anti-PirB 6C1 (BD Biosciences BDB550348). Immunoprecipitate was collected on Protein G beads and liberated by incubation at 85° C. for 5 minutes. Samples were loaded in a 7.5% bis-acrylamide gel and blotted with a 1:1000 dilution of a rabbit antibody raised against the floxed region of PirB. Semi-quantitative densitometry was performed using ImageJ software. Normalized values were then compared with Mann-Whitney U tests. For PirB phosphorylation studies, cortical tissue samples posterior to the infusion site were lysed and pooled together (4 animals/prep), then incubated overnight with anti-phospho-tyrosine-conjugated beads (Cell Signaling 9419S). Immunoprecipitate was collected by centrifugation, then liberated, loaded and blotted against PirB as described above.

Monocular Deprivation or Enucleation:

For monocular enucleation (ME), mice were anesthetized with 1-2.5% isoflurane, one eye was removed and a drop of Vetbond (3M) was placed on eyelids to prevent reopening. Monocular deprivation (MD) was performed either from P70 to P73 or on juvenile (P19) mice for 4 weeks. Mice were anesthetized using isoflurane gas anesthesia (˜1.5-2%). Hair near the eyelid was trimmed, the lid margin was surgically removed and a small drop of ophthalmic cream (Isopto-Max, Alcon Pharma GmbH, or Vetropolycin HC, Pharmaderm Animal Health, NY) was applied to the eye. The eyelid was sutured with 2 to 3 mattress stitches using 6-0 silk (Ethicon). Eye reopening was carried out at P47 under isoflurane anesthesia. The reopened eye was covered with ophthalmic cream again to prevent corneal damage, and was inspected daily to ensure that it did not re-close.

sPirB Protein Production:

To create a soluble PirB mimic, the PirB ectodomain was cloned into a plasmid containing a His tag for purification and a Myc tag for antibody detection (a gift from Bin-Quan Zhuang and Linda Hsieh-Wilson, California Institute of Technology), with a sequence identical to previous publications. HEK 293 cells were transfected using Lipofectamine 2000 (Invitrogen) with a plasmid containing PirB-Myc-His (sPirB) or a control plasmid containing Alkaline Phosphatase-Myc-His. Conditioned media was collected, concentrated with a 50 kDa exclusion column (Millipore), and purified using a HisSpin Trap column (GE Healthcare). Western blots were performed using anti-Myc mouse monoclonal 9E10 (Developmental Studies Hybridoma Bank, Iowa, 9E10), and a polyclonal goat anti-PirB antibody (R&D Systems #AF2754) directed against the extracellular domain. Subsequently, Invitrogen Custom Services produced sPirB in FreeStyle HEK 293 cells and purified it on a nickel column. Aliquots of this commercially produced protein were also assayed for quality with western blotting as described above-results were identical to those shown in FIG. 4B.

Osmotic Minipump Implantations and sPirB Infusion:

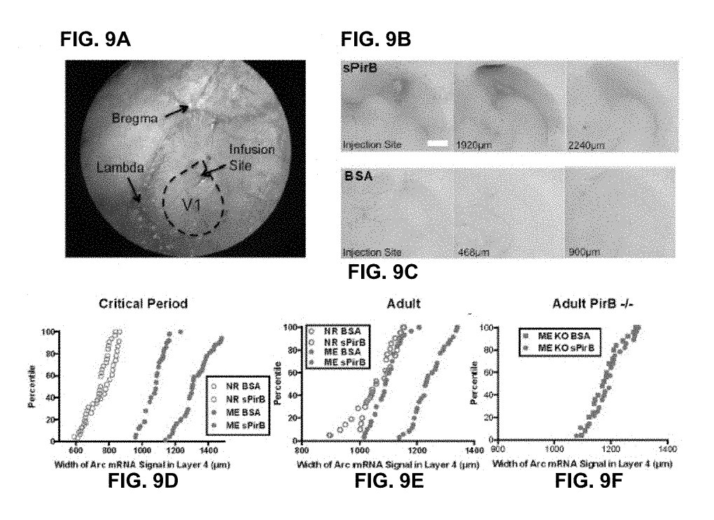

Minipumps (Alzet model 1002; 0.25 μL/hr, 100 μL capacity) were filled the night before surgery and stored in 0.9% saline at 37° C. before implantation. Mice were anesthetized with 1-2.5% isoflurane and positioned on a stereotaxic frame (Kopf). A scalp incision exposed the cranium, and coordinates were zeroed at bregma and lambda to ensure the skull was flat. After a craniotomy, a custom minipump cannula (Plastics One, Roanoke, Va.) with a beveled 30-gauge, 1 mm long needle was implanted. To minimize damage, the minipump cannula was inserted just anterior to visual cortex (2.5 mm lateral and 3 mm posterior to bregma), with the needle bevel facing posterior and its opening located 1 mm below the skull surface, to direct sPirB into visual cortex (FIG. 9A). The cannula base was secured to the skull with Loctite 454 cyanoacrylate glue (Henkel, Westlake, Ohio). The minipump was attached to the cannula via plastic tubing and placed subcutaneously between the scapulae. Minipumps contained either 1 mg/mL PirB-Myc-His, or 1 mg/mL Bovine Serum Albumin (BSA, VWR EM-2930) in 0.1M phosphate buffered saline (PBS). All experiments involved littermate controls and were performed blind to genotype and/or treatment.

sPirB Immunohistochemistry:

Brains were snap-frozen in M1 mounting media (Shandon), and sectioned at 16 μm on a cryostat. Sections were fixed in 4% paraformaldehyde in 0.1M PBS for 30 minutes, washed with PBS, and then blocked in 10% goat serum, 0.3% Triton-X for 1 hour. Sections were incubated overnight at 4° C. with anti-Myc mouse monoclonal 4A6 (Millipore 05-724) at a 1:500 dilution in 2% goat serum and 0.1% Triton-X, then washed, and incubated at room temperature for 1 hour with goat anti-mouse secondary antibody conjugated to alkaline phosphatase (Jackson Immunoresearch 115-055-062) at a 1:1000 dilution in 1% goat serum. Staining was visualized using the colorimetric NBT/BCIP substrate (Roche 11681451001).

Dendritic Spine Analysis:

After minipump infusion, mice were deeply anaesthetized with isoflurane, and the brain was fixed by transcardial perfusion of 0.1M phosphate buffered saline, followed by 4% paraformaldehyde in PBS. 150 μm thick coronal sections were cut on a vibratome (Leica VT1200s), mounted on slides with Prolong Gold with DAPI (Invitrogen) and imaged using two-photon laser scanning microscopy (Prairie Technologies, Middleton, Wis.). As described previously, YFP-labeled pyramidal somata were anatomically localized to L5 in the binocular zone of the visual cortex using a 10× objective, then imaged using a 60× coverslip-corrected water immersion objective (Olympus, NA 1.2) and 4× optical zoom. For apical tuft spines, each L5 cell's apical dendrite was traced to the first branch point in L2/3, and z-stacks were acquired of as much dendritic arbor as was visible in the section (at least 100 μm). For basolateral dendrites, z-stacks were acquired for a single dendrite per cell, again with a minimum length of 100 μm. Spine density was measured in Fiji, a build of ImageJ software (www.fiji.sc, MBF “Image J for Microscopy” collection of plugins by Tony Collins; National Institutes of Health, Bethesda, Md.), using the Cell Counter (Kurt De Vos) and Simple Neurite Tracer (Mark Longair) plugins to count spines and measure dendritic length. All analysis was performed blind to treatment.

Slice Electrophysiology:

As previously described, after brief intracardial perfusion of ice cold ACSF (in mM; 125 NaCl, 26 NaHCO₃, 2.3 KCl, 1.26 KH₂PO₄, 1.3 MgCl₂, 2.5 CaCl₂, and 10 glucose, aerated with 95% O₂/5% CO₂) brains from P70 to P77 male mice that had received minipump infusion of sPirB or BSA were removed and coronal sections (400 μm) including visual cortex were made using a vibratome (Leica VT1000S) in ice-cold NMDG solution (in mM: 135 NMDG, 1 KCl, 1.2 KH2PO4, 1.5 MgCl2, 0.5 CaCl2, 20 choline bicarbonate, and 10 glucose). Sections were transferred to a recovery chamber containing 37° C. ACSF for 30 min, then at room temperature for 30 min before recordings. All recordings were done at 30-32° C. in a chamber with constant ACSF flow. Whole-cell patch clamp recordings were performed from pyramidal neurons in L5 of visual cortex. The recording pipette (2˜4 MΩ) contained Cs+-based internal solution (in mM: 105 CsCl, 20 TEA-Cl, 2 MgCl2, 1 EGTA, 10 HEPES, 3 Mg-ATP, 15 Phosphocreatine, 1 Na-GTP, 5 QX-314, pH 7.4, 280 mOsm). Miniature excitatory postsynaptic currents (mEPSCs) were isolated by applying TTX (1 μM, Sigma), SR95531 (20 μM; Tocris, MO) to block GABA-A receptors and APV (100 μM; Tocris, MO) to block NMDA receptors. Synaptic responses were recorded using an Axopatch 200B amplifier (Molecular Devices, CA), digitized using Digidata 1322A (Axon Instruments, CA) and data acquisition was performed by Clampex 9.2 (Axon Instruments, CA). Data analysis was conducted using MiniAnalysis software (ver. 6.0.7) (Synaptosoft).

Visual Evoked Potential Recordings:

Animals were anesthetized with urethane (0.6-1.2 g/kg, Sigma) and chlorprothixene (5 mg/kg, Sigma), and incisions were treated with lidocaine (2%, Sparhawk Laboratories), then the scalp was exposed and the minipump cannula removed. A plastic headplate was glued to the skull (Loctite), and then surrounded by a layer of dental cement (A-M systems) to form a small enclosure. A ˜1 mm square craniotomy was performed centered over V1 at 3.5 mm posterior to bregma and 3 mm lateral, approximately 1 mm behind the infusion site, and the enclosure was filled with HEPES buffered ACSF (NaCl 138, KCl 3.5, CaCl₂ 2, MgSO₄ 2, glucose 10 and HEPES 10, at pH 7.4, adjusted by 10 M NaOH). A glass pipette filled with ACSF was used to record local field potentials at a depth of 450-600 μm. Recordings were made with a Multiclamp 700B amplifier and sampled at 10 kHz controlled by the Clampex software (Molecular Devices). Responses were averaged over stimulus blocks, and a Matlab algorithm determined the peak response amplitude within a 500-ms window after stimulus onset. Averaged responses were examined, and only those with a characteristic VEP shape were analyzed. Visual acuity was estimated by finding the x-intercept of a semilogarithmic regression of response amplitudes across different spatial frequencies.

Visual Stimuli:

Visual stimuli were generated with a Graphics card in a PC running custom software. Mice viewed a gamma-corrected 7″ LCD monitor (maximal luminance: 250 cd/m₂) with a refresh rate of 75 Hz placed 10 cm away from the left eye. To measure visual acuity, full field monocular stimuli (0° or 90° static sinusoidal gratings) were presented at 1 Hz, with 10 different spatial frequencies ranging from 0.01 to 0.5 cycles/degree. Stimuli were delivered in blocks of 80 randomized repetitions per spatial frequency.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of increasing synaptic connectivity and plasticity the method comprising:
administering to the individual an effective amount of an agent that disrupts PirB function.

2. The method according to claim 1, wherein the disease or disorder is a visual disorder.

3. The method of claim 2, wherein the disorder is amblyopia or glaucoma.

4. The method according to any one of claims 1-3, wherein the agent comprises a PirB/LILRB2 polypeptide.

5. The method according to claim 4, wherein the PirB/LILRB2 polypeptide comprises the six Ig-like domains of PirB or LILRB2.

6. The method according to claim 4, wherein the PirB/LILRB2 polypeptide consists essentially of the six Ig-like domains of PirB or LILRB2.

7. The method according to claim 4, wherein the agent comprises a dimer of PirB/LILRB2 polypeptides.

8. The method according to claim 7, wherein each PirB/LILRB2 polypeptide of the dimer is fused to an Fc domain.

9. The method according to claim 7, wherein each PirB/LILRB2 polypeptide of the dimer comprises the six Ig-like domains of PirB or LILRB2.

10. The method according to claim 7, wherein each PirB/LILRB2 polypeptide of the dimer consists essentially of the six Ig-like domains of PirB or LILRB2.

11. The method according to any one of claims 1-3, wherein the agent is an antibody that binds to amino acid residues within the first or second Ig-like domain of PirB or LILRB2.

12. The method according to any one of claims 1-3, wherein the agent inhibits Aβ oligomer-induced PirB/LILRB2 activation of downstream proteins.

13. The method according to any one of claims 1-3, wherein the agent inhibits PirB/LILRB2 activation of cofilin, PP2A, PP2B or PP2C.

14. The method according to any one of claims 1-3, wherein the method further comprises measuring visual acuity in the subject.


SEQUENCE LISTING (19 sequences)

SEQ ID NO: 1 - protein, 841 aa, Mus musculus
  ref <308>: DATABASE ACCESSION NUMBER: NCBI Reference Sequence: NP_035225
  ref <309>: DATABASE ENTRY DATE: 2015-02-15
  ref <313>: RELEVANT RESIDUES IN SEQ ID NO: (1)..(841)
MSCTFTALLRLGLTLSLWIPVLTGSLPKPILRVQPDSVVSRRTKVTFLCEETIGANEYRLYKDGKLYKTVTKNKQKPENKAEFSFSNVDLSNAGQYRCSYSTQYKSSGYSDLLELVVTGHYWTPSLLAQASPVVTSGGYVTLQCESWHNDHKFILTVEGPQKLSWTQDSQYNYSTRKYHALFSVGPVTPNQRWICRCYSYDRNRPYVWSPPSESVELLVSGNLQKPTIKAEPGSVITSKRAMTIWCQGNLDAEVYFLHNEKSQKTQSTQTLQEPGNKGKFFIPSVTLQHAGQYRCYCYGSAGWSQPSDTLELVVTGIYEYYEPRLSVLPSPVVTAGGNMTLHCASDFPYDKFILTKEDKKFGNSLDTEHISSSGQYRALFIIGPTTPTHTGAFRCYGYYKNAPQLWSVPSALQQILISGLSKKPSLLTHQGHILDPGMTLTLQCFSDINYDRFALHKVGGADIMQHSSQQTDTGFSVANFTLGYVSSSTGGQYRCYGAHNLSSEWSASSEPLDILITGQLPLTPSLSVQPNHTVHSGETVSLLCWSMDSVDTFILSKEGSAQQPLRLKSKSHDQQSQAEFSMSAVTSHLSGTYRCYGAQDSSFYLLSSASAPVELTVSGPIETSTPPPTMSMPLGGLHMYLKALIGVSVAFILFLFIFIFILLRRRHRGKFRKDVQKEKDLQLSSGAEEPITRKGELQKRPNPAAATQEESLYASVEDMQTEDGVELNSWTPPEEDPQGETYAQVKPSRLRKAGHVSPSVMSREQLNTEYEQAEEGQGANNQAAESGESQDVTYAQLCSRTLRQGAAASPLSQAGEAPEEPSVYATLAAARPEAVPKDMEQ

SEQ ID NO: 2 - protein, 40 aa, Homo sapiens
DAEFRHDSGYEVHHQKLVFFAEDVGSNKGAIIGLMVGGVV

SEQ ID NO: 3 - protein, 598 aa, Homo sapiens
  ref <308>: DATABASE ACCESSION NUMBER: NCBI Reference Sequence: NP_005865
  ref <309>: DATABASE ENTRY DATE: 2015-03-15
  ref <313>: RELEVANT RESIDUES IN SEQ ID NO: (1)..(598)
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 4 - protein, 42 aa, Homo sapiens
DAEFRHDSGYEVHHQKLVFAAEDVGSNKGAIIGLMVGGVVIA

SEQ ID NO: 5 - protein, 28 aa, Homo sapiens
DAEFRHDSGYEVHHQAAVFAAEDVGSNK

SEQ ID NO: 6 - protein, 18 aa, Homo sapiens
VHHQKLVFFAEDVGSNKC

SEQ ID NO: 7 - protein, 37 aa, Homo sapiens
DAEFRHDSGYEVHHQKLVFFAEDVGSNKGAIIGLMVG

SEQ ID NO: 8 - protein, 38 aa, Homo sapiens
DAEFRHDSGYEVHHQKLVFFAEDVGSNKGAIIGLMVGG

SEQ ID NO: 9 - protein, 39 aa, Homo sapiens
DAEFRHDSGYEVHHQKLVFFAEDVGSNKGAIIGLMVGGV

SEQ ID NO: 10 - protein, 43 aa, Homo sapiens
DAEFRHDSGYEVHHQKLVFFAEDVGSNKGAIIGLMVGGVVIAT

SEQ ID NO: 11 - protein, 44 aa, Homo sapiens
DAEFRHDSGYEVHHQKLVFFAEDVGSNKGAIIGLMVGGVVIATV

SEQ ID NO: 12 - protein, 45 aa, Homo sapiens
DAEFRHDSGYEVHHQKLVFFAEDVGSNKGAIIGLMVGGVVIATVI

SEQ ID NO: 13 - protein, 46 aa, Homo sapiens
DAEFRHDSGYEVHHQKLVFFAEDVGSNKGAIIGLMVGGVVIATVIV

SEQ ID NO: 14 - protein, 47 aa, Homo sapiens
DAEFRHDSGYEVHHQKLVFFAEDVGSNKGAIIGLMVGGVVIATVIVI

SEQ ID NO: 15 - protein, 48 aa, Homo sapiens
DAEFRHDSGYEVHHQKLVFFAEDVGSNKGAIIGLMVGGVVIATVIVIT

SEQ ID NO: 16 - protein, 49 aa, Homo sapiens
DAEFRHDSGYEVHHQKLVFFAEDVGSNKGAIIGLMVGGVVIATVIVITL

SEQ ID NO: 17 - protein, 55 aa, Homo sapiens
DAEFRHDSGYEVHHQKLVFFAEDVGSNKGAIIGLMVGGVVIATVIVITLVMLKKK

SEQ ID NO: 18 - protein, 39 aa, Homo sapiens
AEFRHDSGYEVHHQKLVFFAEDVGSNKGAIIGLMVGGVV

SEQ ID NO: 19 - protein, 38 aa, Homo sapiens
EFRHDSGYEVHHQKLVFFAEDVGSNKGAIIGLMVGGVV